(19) United States
(12) Patent Application Publication
(10) Pub. No.: US 2014/0364601 A1
(43) Pub. Date: Dec. 11, 2014

(54) PROCESS

(71) Applicants: Neil Barnwell, Macclesfield (GB); Philip Cornwall, Macclesfield (GB); Duncan Michael Gill, Macclesfield (GB); Gareth P. Howell, Macclesfield (GB); Rebecca Elizabeth Meadows, Macclesfield (GB); Andiappan Murugan, Hebbal (IN); Philip O'Keefe, Macclesfield (GB); John Singleton, Macclesfield (GB); Jane Withnall, Macclesfield (GB); Eric Merifield, Macclesfield (GB); Christopher William Mitchell, Macclesfield (GB); Zakariya Mohamed Patel, Macclesfield (GB); James Barry Rose, Macclesfield (GB)

(72) Inventors: Neil Barnwell, Macclesfield (GB); Philip Cornwall, Macclesfield (GB); Duncan Michael Gill, Macclesfield (GB); Gareth P. Howell, Macclesfield (GB); Rebecca Elizabeth Meadows, Macclesfield (GB); Andiappan Murugan, Hebbal (IN); Philip O'Keefe, Macclesfield (GB); John Singleton, Macclesfield (GB); Jane Withnall, Macclesfield (GB); Eric Merifield, Macclesfield (GB); Christopher William Mitchell, Macclesfield (GB); Zakariya Mohamed Patel, Macclesfield (GB); James Barry Rose, Macclesfield (GB)

(73) Assignee: ASTRAZENECA AB, Sodertalje (SE)

(21) Appl. No.: 14/117,259

(22) PCT Filed: May 11, 2012
(86) PCT No.: PCT/GB12/51037

§ 371 Date: Aug. 21, 2014

(30) Foreign Application Priority Data
May 13, 2011 (GB) 1107985.2

(51) Int. Cl. C07D 498/10; C07C 309/08; C07D 277/68; C07C 55/07

(52) U.S. Cl. CPC C07D 498/10; C07C 55/07; C07C 309/08; C07D 277/68
USPC 544/71; 562/41; 548/173

(57) ABSTRACT

Processes for the preparation of the compound of formula (II) and intermediate compounds for use in the processes.

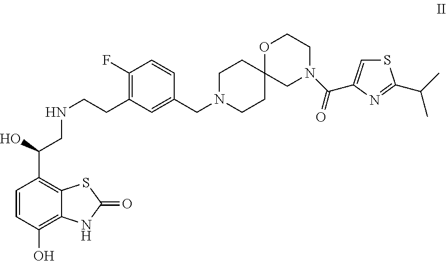 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

The present invention relates to processes for the preparation of chemical compounds that have MABA activity and intermediates for use in such preparations.

The first-line treatment for a variety of pulmonary disorders including chronic obstructive pulmonary disease (COPD) and asthma is through the use of bronchodilators. Muscarinic-receptor antagonists (anti-cholinergics) are bronchodilators that exert their efficacy by reducing vagal cholinergic tone, the main reversible component of airway constriction in COPD. β-adrenoceptor agonists are also bronchodilators due to their ability to functionally antagonise the bronchoconstrictor responses to a range of mediators, including acetylcholine.

In addition to improving lung function, these agents improve dyspnoea (breathlessness), quality of life, exercise tolerance and they reduce exacerbations. A number of clinical studies have demonstrated that combined administration of an anti-cholinergic and a β₂-receptor agonist is more efficacious than either of the individual components (van Noord, J. A., Aumann, J-L., Janssens, E., Smeets, J. J., Verhaert, J., Disse, B., Mueller, A. & Cornelissen, P. J. G., 2005. “Comparison of tiotropium once daily, formoterol twice daily and both combined once daily in patients with COPD”, Eur. Respir. J., vol 26, pp 214-222.). A single molecule possessing activities at muscarinic and β₂-receptors (MABA) may provide additional benefits to COPD patients in terms of efficacy and side-effect profile over either single agent. Moreover, a molecule possessing dual activity may also offer benefits in terms of ease-of-use and patient compliance over co-administration of the single therapies. A single agent may also be beneficial from the perspective of formulation compared to two separate compounds, also offering the potential, if combined with another therapeutic agent, for triple action therapies.

The compound of Formula II and pharmaceutically acceptable salts thereof and processes for their preparation are disclosed in PCT patent application, publication no. WO2009/098448.

We have now devised novel processes for the preparation of the compound of formula II.

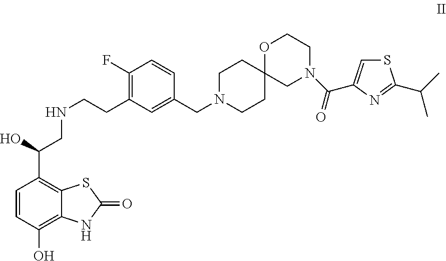 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

### Route 1

In a first aspect of the invention we provide a process for the preparation of the compound of formula II

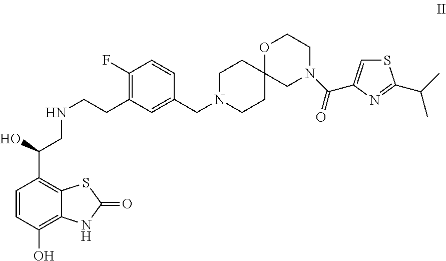 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

which comprises reaction of the compound of formula III or any other suitable alternate salt thereof

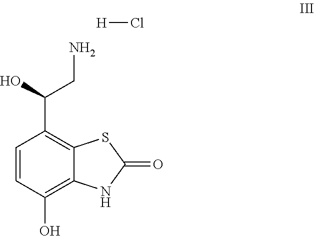

and the compound of formula V

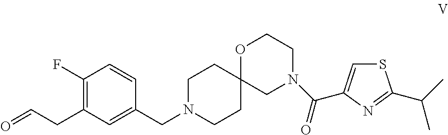 [SMILES: CC(C)c1nc(C(=O)N2CCOC3(CCN(Cc4ccc(F)c(CC=O)c4)CC3)C2)cs1]

in a suitable solvent, for example N-methylpyrrolidinone or dimethylformamide, at a suitable temperature, for example in the range 10 to 70° C. and under reductive conditions such as hydrogen in the presence of a metal catalyst such as Iridium, so as to give the compound of formula II.

We have found that use of Iridium catalysed reductive amination as above provides the compound of formula II in about 70-80% yield. This compares to typically 30-50% yield when using standard reductive amination conditions such as for example sodium triacetoxyborohydride or palladium on charcoal. Moreover the quality of material that is obtained from the Iridum catalysed reductive amination is sufficient to allow the compound of formula I (see Scheme 1 below) to be crystallised directly from the reaction mixture post aqueous work-up.

The compound of formula III is prepared from the compound IV

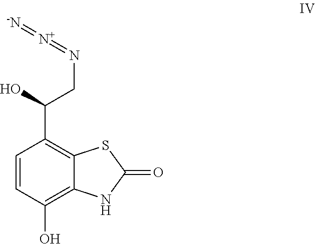

where in IV is dissolved in a suitable solvent, for example methanol, in the presence of an acid for example aqueous hydrochloric acid; at a temperature, for example in the range 0 to 70° C. under reductive conditions such as hydrogen in the presence of a metal catalyst. The compound of formula IV may be prepared using the method disclosed in WO-2009/098448 in Example 1 on page 51.

The compound of formula V is conveniently prepared from the compound of formula VI or any other suitable alternate salt there of

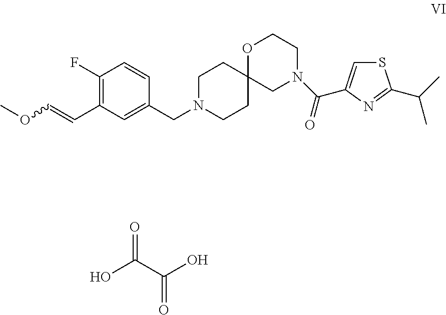 [SMILES: COC=Cc1cc(CN2CCC3(CC2)CN(C(=O)c2csc(C(C)C)n2)CCO3)ccc1F ; O=C(O)C(=O)O]

via the addition of VI to a suitable acid, for example hydrochloric acid at a temperature, for example in the range 10 to 70° C.

The compound of formula VI is prepared from the compound of formula VII

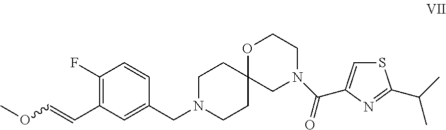 [SMILES: COC=Cc1cc(CN2CCC3(CC2)CN(C(=O)c2csc(C(C)C)n2)CCO3)ccc1F]

in a suitable solvent, for example methyl tetrahydrofuran; at a temperature, for example in the range 10 to 60° C., via the addition of oxalic acid.

The compound of formula VII is prepared by reaction of the compound of formula VIII

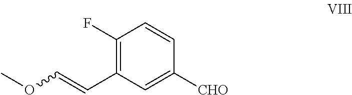

or compound VIIIa

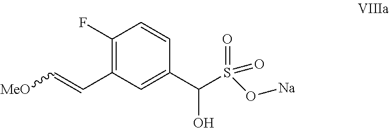

with the compound of formula IX or any other suitable alternate salt there of

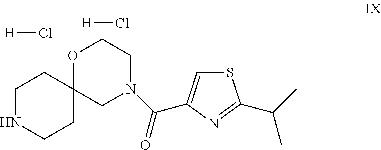

where in compound IX in a suitable solvent for example methyl tetrahydrofuran or dichloromethane; in the presence of a base, for example sodium hydroxide or triethylamine; is reacted with VIII or VIIIa (after liberation of parent aldehyde VIII via treatment with base e.g. sodium bicarbonate) in the presence of a reducing agent for example sodium triacetoxyborohydride.

The compound of formula VIIIa is prepared from the compound of formula VIII

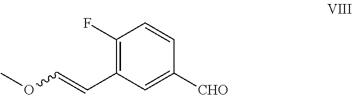

via reaction with sodium metabisulfite in a suitable solvent e.g. ethanol at a temperature between 0-70° C.

The compound of formula VIII is conveniently prepared using the method disclosed in WO 2009/098448 in Example 47E on page 202.

The compound of formula IX is prepared by reaction of the compound of formula X

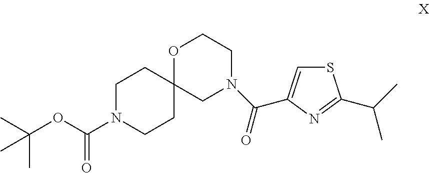 [SMILES: CC(C)c1nc(C(=O)N2CCOC3(CCN(C(=O)OC(C)(C)C)CC3)C2)cs1]

in a suitable solvent for example isopropyl alcohol; by addition of a suitable acid, for example hydrochloric acid in isopropyl alcohol.

The compound of formula X is prepared by reaction of the compound of formula XI

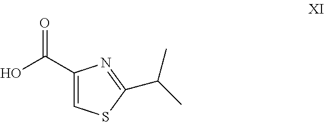

and the compound of formula XII or any other suitable alternate salt there of

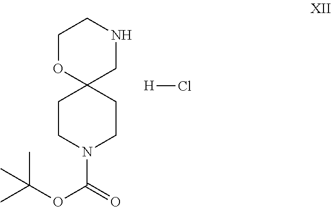

in a suitable solvent for example methyl tetrahydrofuran; in the presence of a base, for example triethylamine; by the addition of coupling reagent for example 2-propanephosphonic acid anhydride (T3P).

The compound of formula XI may be obtained using the process set out in WO-1999/038862 (page 37, preparation 4).

The compound of formula XII may be obtained from WuXi Pharma Tech.

The above route is conveniently illustrated in Scheme 1.

### Route 2

In a further aspect we provide a process for the preparation of the compound of formula II

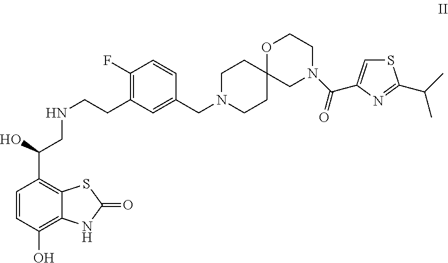 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

which process comprises reaction of the compound of formula XX

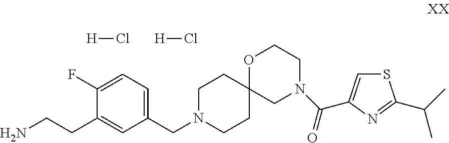

or any other suitable alternate salt (or the neutral, parent amine) there of with the compound of formula XIV

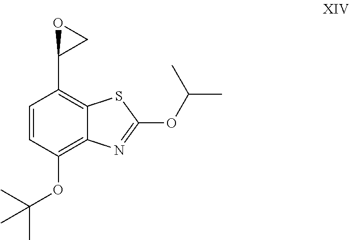 [SMILES: CC(C)Oc1nc2c(OC(C)(C)C)ccc([C@@H]3CO3)c2s1]

in a suitable solvent for example N,N-dimethylformamide, N,N-dimethylacetamide, dimethylsulfoxide or 4-methyl-2-pentanol; in the presence of a base (not required when using the neutral, parent amine XX) for example sodium bis(trimethylsilyl)amide or potassium carbonate; at a temperature, for example in the range 20 to 150° C.

to give the compound of formula XIII

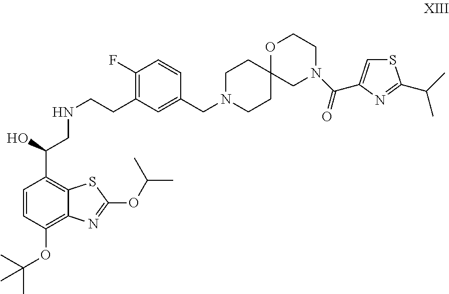 [SMILES: CC(C)Oc1nc2c(OC(C)(C)C)ccc([C@@H](O)CNCCc3cc(CN4CCC5(CC4)CN(C(=O)c4csc(C(C)C)n4)CCO5)ccc3F)c2s1]

followed by deprotection so as to give the compound of formula II.

We have found that simple benzothiazolones of the type XIV require protecting groups (O,O′ or O, N) to increase stability allowing isolation and subsequent chemical manipulation. We have unexpectedly found that the specific combination of t-butyl and isopropyl groups as shown, is stable enough to allow the chemistry used in formation of the parent benzothiazolone and epoxide derivative; the subsequent epoxide opening can be achieved and these specific protecting groups can be easily removed to allow formation of the compound of formula II or its salt I.

The compound of formula XIV is conveniently prepared and used in-situ from the compound of formula XV

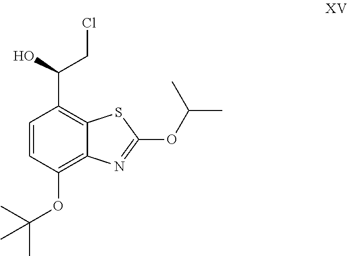 [SMILES: CC(C)Oc1nc2c(OC(C)(C)C)ccc([C@@H](O)CCl)c2s1]

in a suitable solvent, N,N-dimethylformamide, N,N-dimethylacetamide, dimethylsulfoxide or 4-methyl-2-pentanol by the addition of a base; for example sodium hexamethyldisilazide or potassium carbonate; at a temperature, for example in the range 20 to 90° C.

The compound of formula XIV may be prepared and isolated from the compound of formula XV

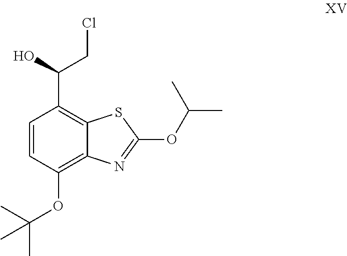 [SMILES: CC(C)Oc1nc2c(OC(C)(C)C)ccc([C@@H](O)CCl)c2s1]

according to the process set out in WO-2004/016601 (preparation 30, page 28). The compound of formula XV may be prepared from the compound of formula XVI

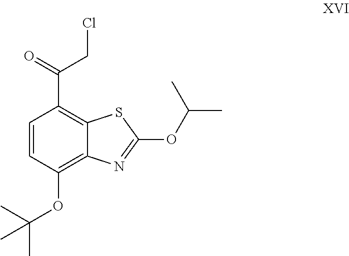 [SMILES: CC(C)Oc1nc2c(OC(C)(C)C)ccc(C(=O)CCl)c2s1]

using the method disclosed in WO-2004/016601 (page 27, preparation 29).

The compound of formula XV may also be prepared from the compound of formula XVI

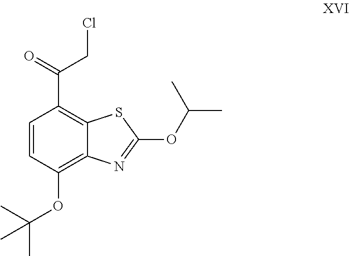 [SMILES: CC(C)Oc1nc2c(OC(C)(C)C)ccc(C(=O)CCl)c2s1]

by treatment with a hydrogen source e.g. H₂ or triethylamine/formic acid in the presence of a suitable metal/homochiral ligand complex e.g. [(S,S)-TsDpen-Ru(p-cymene)Cl] in a suitable solvent e.g. acetonitrile or dichloromethane at a temperature between 0 and 100° C.

The compound of formula XVI may be prepared from the compound of formula XVII

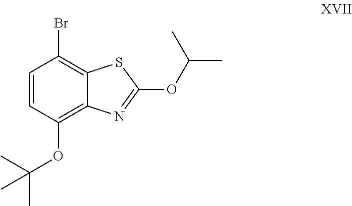 [SMILES: CC(C)Oc1nc2c(OC(C)(C)C)ccc(Br)c2s1]

in a suitable solvent for example methyl t-butyl ether; by the addition of a base for example n-butyllithium; at a temperature for example −80 to 0° C.; followed by the addition of a suitable chloroacetyl compound for example 2-chloro-N-methoxy-N-methyl acetamide or chloroacetylchloride or it may be obtained directly from the compound of formula XIX

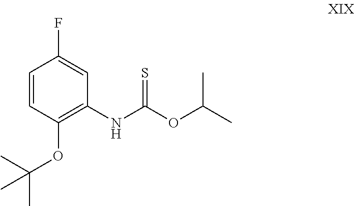 [SMILES: CC(C)OC(=S)Nc1cc(F)ccc1OC(C)(C)C]

(as set out in WO-2004/016601, page 27, preparation 28)

The compound of formula XVII is conveniently prepared from the compound of formula XVIII

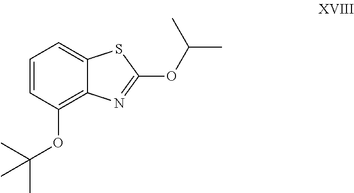 [SMILES: CC(C)Oc1nc2c(OC(C)(C)C)cccc2s1]

in a suitable solvent for example 2-methyl tetrahydrofuran by the addition of a electrophilic brominating reagent for example N-bromosuccinimide; at a temperature, for example in the range 0 to 90° C.

The compound of formula XVIII is conveniently prepared from the compound of formula XIX

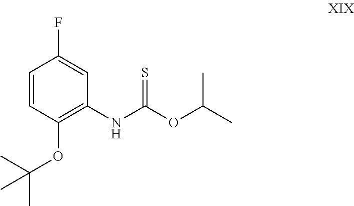 [SMILES: CC(C)OC(=S)Nc1cc(F)ccc1OC(C)(C)C]

in a suitable solvent for example 2-methyl-tetrahydrofuran; by addition to a base for example a combination of n-butyllithium and diisopropylamine (lithium diisopropylamide) or t-butyllithium; at a temperature of for example −80 to 0° C.

The compound of formula XIX is conveniently prepared using the process disclosed in WO 2004/016601 (preparation 9, page 23).

The compound of formula XX is conveniently prepared from the compound of formula XXI

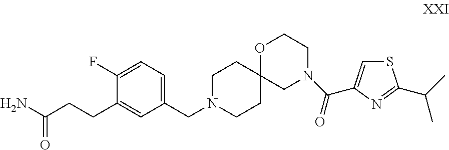 [SMILES: CC(C)c1nc(C(=O)N2CCOC3(CCN(Cc4ccc(F)c(CCC(N)=O)c4)CC3)C2)cs1]

in a suitable solvent for example acetonitrile by the addition of a hypervalent iodine compound for example [bis(trifluoroacetoxy)iodo]benzene or a similar oxidising agent to carry out what is known as a Hofmann rearragnement; at a temperature, for example in the range 20 to 90° C.; and treatment of the resulting mixture with an acid for example sulphuric acid. The dihydrochloride salt is prepared via addition of a form of hydrochloric acid for example 15% hydrochloric acid in isopropyl alcohol.

The compound of formula XXI is conveniently prepared from the compound of formula XXII

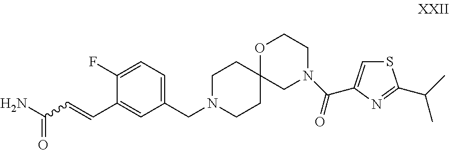 [SMILES: CC(C)c1nc(C(=O)N2CCOC3(CCN(Cc4ccc(F)c(C=CC(N)=O)c4)CC3)C2)cs1]

in a suitable solvent for example methanol by the addition of a metal catalyst for example 10% Pd/C and subject to a hydrogen atmosphere.

The compound of formula XXII is conveniently prepared from the compound of formula XXIII

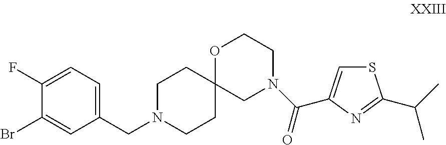 [SMILES: CC(C)c1nc(C(=O)N2CCOC3(CCN(Cc4ccc(F)c(Br)c4)CC3)C2)cs1]

in a suitable solvent for example acetonitrile by the addition of acrylamide in the presence of a metal catalyst for example dichlorobis(tri-ortho-tolylphosphine) palladium (II) [Pd-115] and a base for example diisopropylethylamine to effect what is known as a Heck reaction. The compound of formula XXIII is prepared by reaction of the compound of formula XXIV

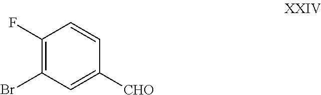

with the compound of formula IX or any other suitable alternate salt there of

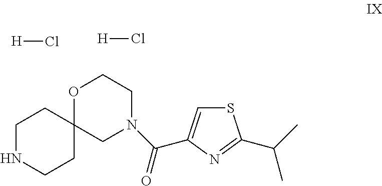

in a suitable solvent for example dichloromethane; in the presence of a base for example diisopropylethylamine; by the addition of reducing agent for example sodium triacetoxyborohydride.

The compound of formula IX is conveniently prepared from the compound of formula X

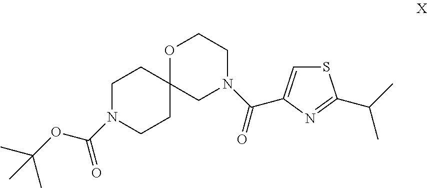 [SMILES: CC(C)c1nc(C(=O)N2CCOC3(CCN(C(=O)OC(C)(C)C)CC3)C2)cs1]

wherein convenient reaction conditions are disclosed hereinbefore.

The compound of formula X is conveniently prepared from the reaction of the compound of formula XI

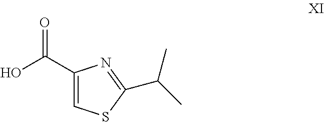

with the compound of formula XII

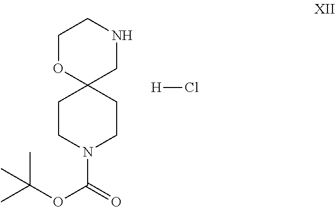

wherein convenient reaction conditions are disclosed hereinbefore.

The compound of formula XI may be obtained using the process set out in WO-1999/038862 (page 37, preparation 4).

The compound of formula XII may be obtained from WuXi Pharma Tech.

The compound of formula XXIV may be obtained from Sigma Aldrich.

The above route is conveniently illustrated in Scheme 2

### Route 3

According to a further aspect of the invention we provide a process for preparing the compound of formula II

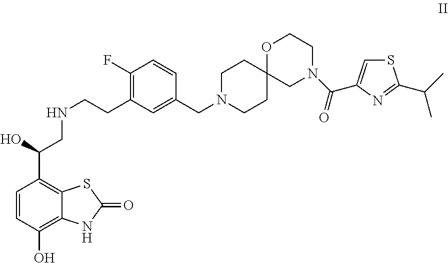 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

which process comprises reacting the compound of formula XVI

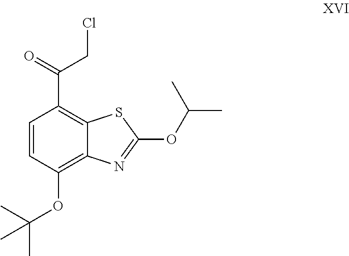 [SMILES: CC(C)Oc1nc2c(OC(C)(C)C)ccc(C(=O)CCl)c2s1]

with the compound of formula XXVII

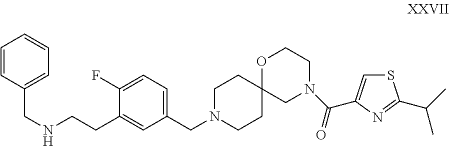 [SMILES: CC(C)c1nc(C(=O)N2CCOC3(CCN(Cc4ccc(F)c(CCNCc5ccccc5)c4)CC3)C2)cs1]

in a suitable solvent for example N-methylpyrrolidinone in the presence of a base for example diisopropylamine and a source of iodide for example sodium iodide to give the compound of formula XXVI

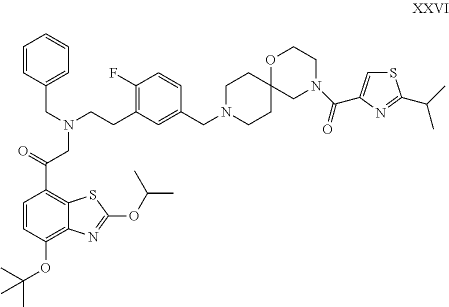 [SMILES: CC(C)Oc1nc2c(OC(C)(C)C)ccc(C(=O)CN(CCc3cc(CN4CCC5(CC4)CN(C(=O)c4csc(C(C)C)n4)CCO5)ccc3F)Cc3ccccc3)c2s1]

which is then reduced in a suitable alcoholic solvent for example isopropyl alcohol; over a time for example over 1-10 hrs; under transfer hydrogenation conditions for example a mixture of formic acid and triethylamine; using a homochiral transition metal/ligand complex for example [(S,S)-teth-TsDpen-RuCl] to give the compound of formula XXV

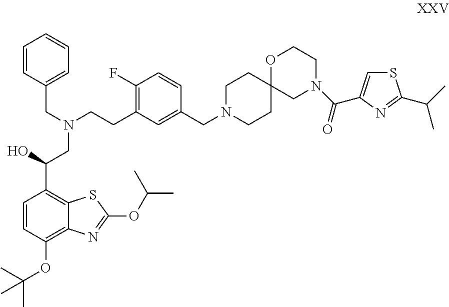 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1]

which is then deprotected in a suitable solvent for example formic acid in the presence of a metal catalyst for example palladium black so as to give the compound of formula II.

We have found that the benzyl protection in the compound of formula XXI is key to preventing impurity formation in the production of the compound of formula XXVI. Whilst we do not wish to be limited by theoretical considerations the benzyl, t-butyl and isopropyl groups are key to providing the necessary bulk around the carbonyl group located adjacent to the benzothiazole, allowing the subsequent reduction to the compound of formula XIV to proceed stereoselectively by addition to a complex chiral reduction catalyst. We believe the choice of the protecting groups benzyl, t-butyl and isopropyl groups is key, not only for the reduction, but for the assembly of the benzathiazolone ring and ease of deprotection to form the compound of formula II or its salt I.

The compound of formula XVI is prepared from the compound of formula XVII

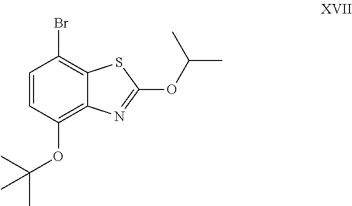 [SMILES: CC(C)Oc1nc2c(OC(C)(C)C)ccc(Br)c2s1]

wherein convenient reaction conditions are disclosed hereinbefore.

The compound of formula XVII is prepared from the compound of formula XVIII

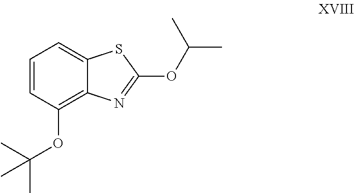 [SMILES: CC(C)Oc1nc2c(OC(C)(C)C)cccc2s1]

wherein convenient reaction conditions are disclosed hereinbefore.

The compound of formula XVIII is prepared from the compound of formula XIX

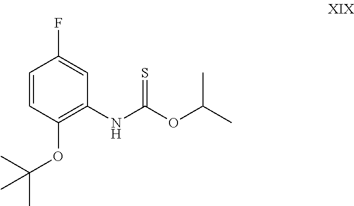 [SMILES: CC(C)OC(=S)Nc1cc(F)ccc1OC(C)(C)C]

wherein convenient reaction conditions are disclosed hereinbefore.

The compound of formula XIX is conveniently prepared using the process disclosed in WO 2004/016601 (preparation 9, page 23).

The compound of formula XXVII is conveniently prepared from the compound of formula XX or any other suitable alternate salt there of (or the neutral, parent amine)

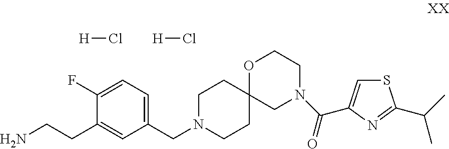 [SMILES: Cl ; Cl ; CC(C)c1nc(C(=O)N2CCOC3(CCN(Cc4ccc(F)c(CCN)c4)CC3)C2)cs1]

in a suitable solvent for example ethanol; by the addition of benzylamine, a metal catalyst; for example iridium on calcium carbonate; the mixture then being subjected to a hydrogenation; for example 1-10 bar of a hydrogen atmosphere; at a temperature for example 10 to 60° C.

The compound of formula XX is conveniently prepared from the compound of formula XXI

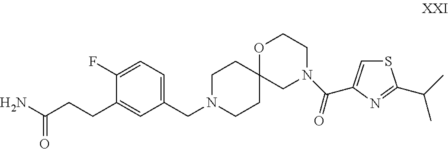 [SMILES: CC(C)c1nc(C(=O)N2CCOC3(CCN(Cc4ccc(F)c(CCC(N)=O)c4)CC3)C2)cs1]

wherein convenient reaction conditions are disclosed hereinbefore.

The compound of formula XXI is conveniently prepared from the compound of formula XXII

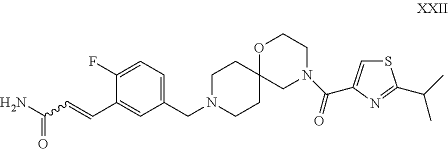 [SMILES: CC(C)c1nc(C(=O)N2CCOC3(CCN(Cc4ccc(F)c(C=CC(N)=O)c4)CC3)C2)cs1]

wherein convenient reaction conditions are disclosed hereinbefore.

The compound of formula XXII is conveniently prepared from the compound of formula XXIII

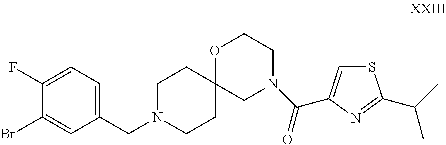 [SMILES: CC(C)c1nc(C(=O)N2CCOC3(CCN(Cc4ccc(F)c(Br)c4)CC3)C2)cs1]

wherein convenient reaction conditions are disclosed hereinbefore.

The compound of formula XXIII is conveniently prepared by reaction of the compound of formula IX

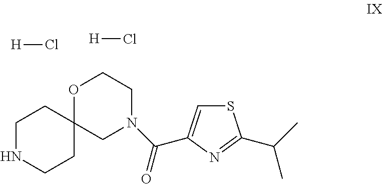

with the compound of formula XXIV

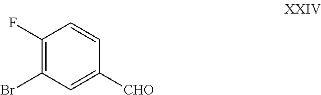

wherein convenient reaction conditions are disclosed hereinbefore.

The compound of formula IX is conveniently prepared from the compound of formula X

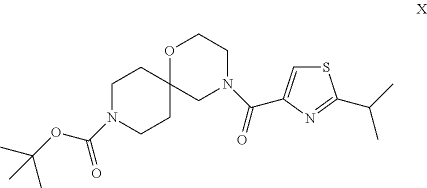 [SMILES: CC(C)c1nc(C(=O)N2CCOC3(CCN(C(=O)OC(C)(C)C)CC3)C2)cs1]

wherein convenient reaction conditions are disclosed hereinbefore.

The compound of formula X is conveniently prepared from the reaction of the compound of formula XI

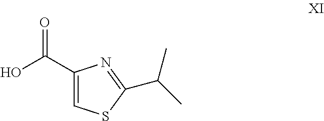

with the compound of formula XII or any other suitable alternate salt there of

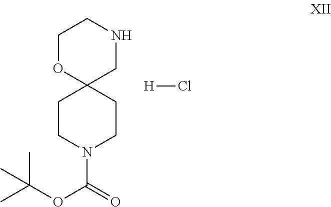

wherein convenient reaction conditions are disclosed hereinbefore.

The compound of formula XI may be obtained using the process set out in WO-1999/038862 (page 37, preparation 4).

The compound of formula XII may be obtained from WuXi Pharma Tech.

The compound of formula XXIV may be obtained from Sigma Aldrich.

The above route is conveniently illustrated in Scheme 3.

### Route 4

According to a further aspect of the invention we provide a process for the preparation of the compound of formula II which process comprises reaction of a compound of formula XXIII

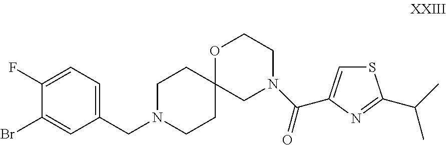 [SMILES: CC(C)c1nc(C(=O)N2CCOC3(CCN(Cc4ccc(F)c(Br)c4)CC3)C2)cs1]

in a suitable solvent for example 2-methyltetrahydrofuran, N-methylpyrrolidinone; by the addition of t-butylvinyl ether; a metal catalyst for example palladium (II) acetate; and ligand/phase transfer catalyst/base combination for example dicyclohexylmethyl amine, tetrabutylammonium bromide or tetrabutylammonium acetate to give a compound of formula XXVIII

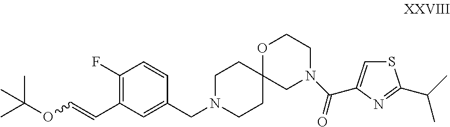 [SMILES: CC(C)c1nc(C(=O)N2CCOC3(CCN(Cc4ccc(F)c(C=COC(C)(C)C)c4)CC3)C2)cs1]

which is then converted to a compound of formula V

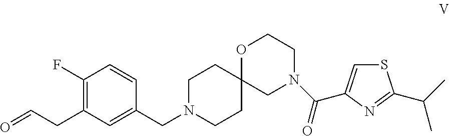 [SMILES: CC(C)c1nc(C(=O)N2CCOC3(CCN(Cc4ccc(F)c(CC=O)c4)CC3)C2)cs1]

via addition to a suitable acid for example hydrochloric acid; at a temperature for example in the range 10 to 70° C., which is then reacted with the compound of formula III

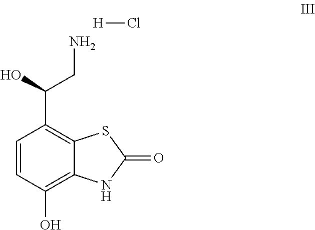

or any alternative salt thereof, in a suitable solvent for example 2-methyltetrahydrofuran and/or N-methylpyrrolidinone; under hydrogenation conditions for example, hydrogen 1-10 bar; in the presence of a metal catalyst or boron based reducing agent e.g. sodium triacetoxyborohydride so as to give the compound of formula II.

We have found that in the above process, the compound of formula XXIII acts as a point of control in that it can isolated as a solid. For the subsequent Heck reaction, most of the literature indicates that an unusable branched regioisomer will predominate or at best an unfavourable mixture will result. however some literature indicates that vinyl ethers can give linear products. Whilst we don't want to be bound by theoretical considerations, the subsequent ease of hydrolysis of XXVIII may allow better access to the unstable aldehyde V.

The compound of formula III is conveniently prepared from the compound of formula IV

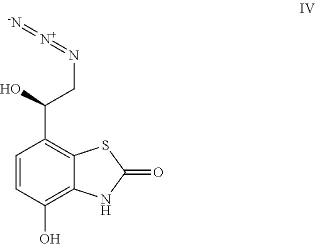

wherein convenient reaction conditions are disclosed hereinbefore.

The compound of formula III may also be prepared using the method disclosed in WO2007027134 in Example 1 on page 47.

The compound of formula XXIII is conveniently prepared by reaction of the compound of formula IX or any other suitable alternate salt there of

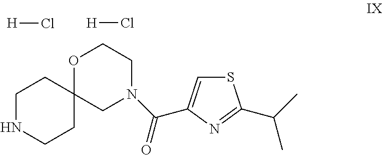

with the compound of formula XXIV

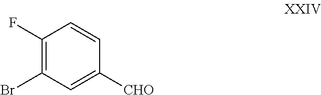

wherein convenient reaction conditions are disclosed hereinbefore.

The compound of formula IX is conveniently prepared from the compound of formula X

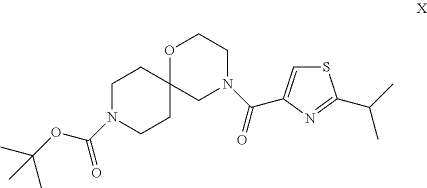 [SMILES: CC(C)c1nc(C(=O)N2CCOC3(CCN(C(=O)OC(C)(C)C)CC3)C2)cs1]

wherein convenient reaction conditions are disclosed hereinbefore.

The compound of formula X is conveniently prepared from the reaction of the compound of formula XI

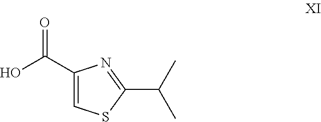

with the compound of formula XII or any other suitable alternate salt there of

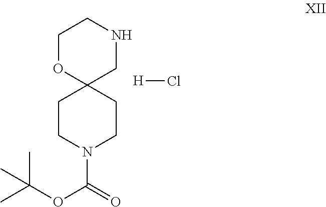

wherein convenient reaction conditions are disclosed hereinbefore.

The compound of formula XI may be obtained using the process set out in WO-1999/038862 (page 37, preparation 4).

The compound of formula XII may be obtained from WuXi Pharma Tech.

The compound of formula XXIV may be obtained from Sigma Aldrich.

The above route is conveniently illustrated in Scheme 4

### Route 5

In a further aspect of the invention we provide a process for the preparation of the compound of formula II which process comprises reaction of a compound of formula XX or any other suitable alternate salt there of (or the neutral, parent amine)

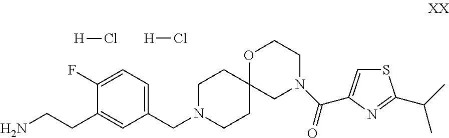

with the compound of formula XXIX

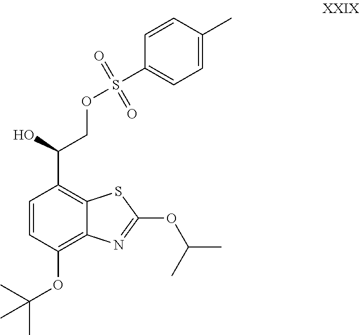 [SMILES: Cc1ccc(S(=O)(=O)OC[C@H](O)c2ccc(OC(C)(C)C)c3nc(OC(C)C)sc23)cc1]

in a suitable solvent for example N,N-dimethylformamide, N,N-dimethylaceamide, dimethylsulfoxide or 4-methyl-2-pentanol; in the presence of a base for example sodium bis (trimethylsilyl)amide; at a temperature, for example in the range 20 to 150° C. to give a compound of the compound of formula XIII

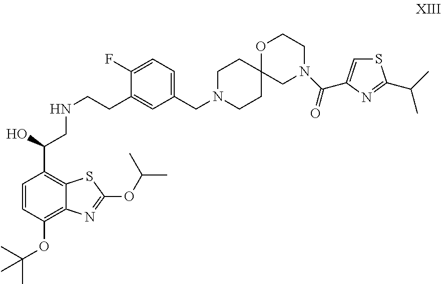 [SMILES: CC(C)Oc1nc2c(OC(C)(C)C)ccc([C@@H](O)CNCCc3cc(CN4CCC5(CC4)CN(C(=O)c4csc(C(C)C)n4)CCO5)ccc3F)c2s1]

followed by deprotection, wherein convenient reaction conditions are disclosed hereinbefore, to give a compound of formula II.

We have found that simple benzothiazolones of the type XIV require protecting groups (O,O′ or O, N) to increase stability allowing isolation and subsequent chemical manipulation. We have unexpectedly found that the specific combination of t-butyl and isopropyl groups as shown, is stable enough to allow the chemistry used in formation of the parent benzothiazolone and epoxide derivative; the subsequent epoxide opening can be achieved and these specific protecting groups can be easily removed to allow formation of the compound of formula II or its salt I.

The compound of formula XX is conveniently prepared from the compound of formula XXI

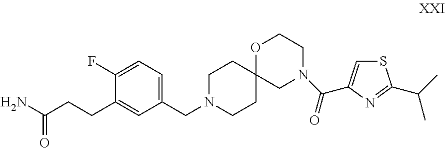 [SMILES: CC(C)c1nc(C(=O)N2CCOC3(CCN(Cc4ccc(F)c(CCC(N)=O)c4)CC3)C2)cs1]

wherein convenient reaction conditions are disclosed hereinbefore.

The compound of formula XXI is conveniently prepared from the compound of formula XXII

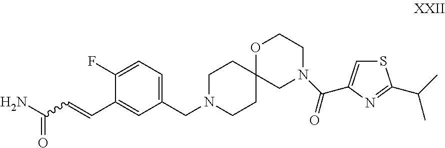 [SMILES: CC(C)c1nc(C(=O)N2CCOC3(CCN(Cc4ccc(F)c(C=CC(N)=O)c4)CC3)C2)cs1]

wherein convenient reaction conditions are disclosed hereinbefore.

The compound of formula XXII is conveniently prepared from the compound of formula XXIII

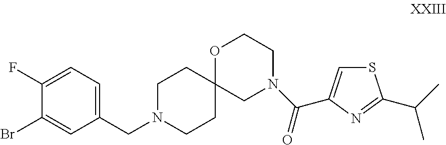 [SMILES: CC(C)c1nc(C(=O)N2CCOC3(CCN(Cc4ccc(F)c(Br)c4)CC3)C2)cs1]

wherein convenient reaction conditions are disclosed hereinbefore.

The compound of formula XXIII is conveniently prepared by reaction of the compound of formula IX

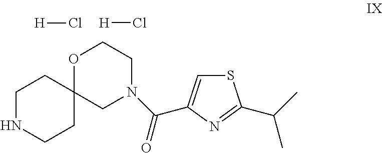

with the compound of formula XXIV

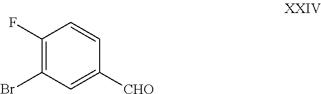

wherein convenient reaction conditions are disclosed hereinbefore.

The compound of formula IX is conveniently prepared from the compound of formula X

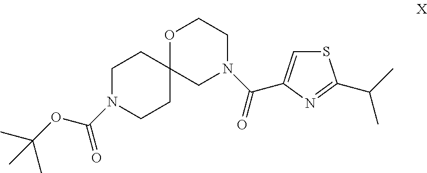 [SMILES: CC(C)c1nc(C(=O)N2CCOC3(CCN(C(=O)OC(C)(C)C)CC3)C2)cs1]

wherein convenient reaction conditions are disclosed hereinbefore.

The compound of formula X is conveniently prepared from the reaction of the compound of formula XI

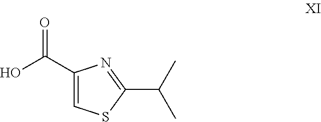

with the compound of formula XII or any other suitable alternate salt there of

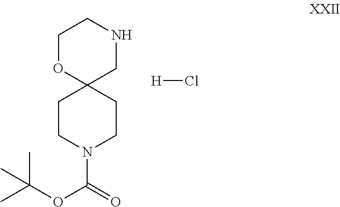

wherein convenient reaction conditions are disclosed hereinbefore.

The compound of formula XI may be obtained using the process set out in WO-1999/038862 (page 37, preparation 4).

The compound of formula XII may be obtained from WuXi Pharma Tech.

The compound of formula XXIV may be obtained from Sigma Aldrich.

The compound of formula XXIX is conveniently prepared from the compound of formula XXX

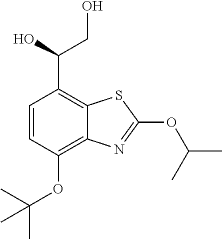 [SMILES: CC(C)Oc1nc2c(OC(C)(C)C)ccc([C@@H](O)CO)c2s1]

in a suitable solvent for example dichloromethane; in the presence of a suitable base for example triethylamine; by the addition of a tosylating agent for example tosyl chloride or tosyl triflate; at a suitable reaction temperature for example −10 to 30° C.

The compound of formula XXX is conveniently prepared from the compound of formula XXXI

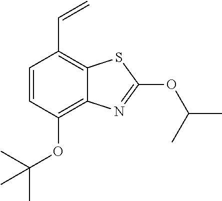 [SMILES: C=Cc1ccc(OC(C)(C)C)c2nc(OC(C)C)sc12]

in a suitable solvent for example tert-butanol; by its addition to a solution of AD-mix-β and methanesulfonamide in water; at a suitable reaction temperature for example −10 to 30° C.

The compound of formula XXXI is conveniently prepared from the compound of formula XVII

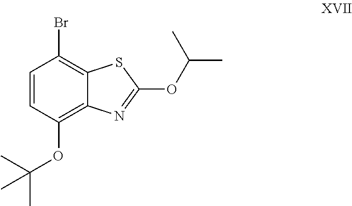 [SMILES: CC(C)Oc1nc2c(OC(C)(C)C)ccc(Br)c2s1]

in a suitable solvent for example acetonitrile; by addition to a mixture of a palladium catalyst, a base and a vinyl synthon, as known by a skilled person to produce the desired Heck coupling; for example Dichloro [1,1′ bis(di-tert-butylphosphino)]ferrocene palladium (II) [Pd-118], potassium carbonate and 4,4,5,5,-tetramethyl-2-vinyl-1,3,2-dioxaborolane.

The compound of formula XVII is conveniently prepared from the compound of formula XVIII

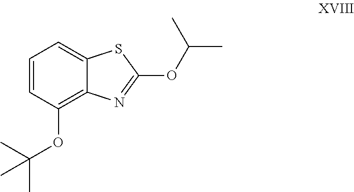 [SMILES: CC(C)Oc1nc2c(OC(C)(C)C)cccc2s1]

wherein convenient reaction conditions are disclosed hereinbefore.

The compound of formula XVIII is conveniently prepared from the compound of formula XIX

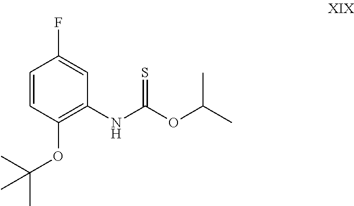 [SMILES: CC(C)OC(=S)Nc1cc(F)ccc1OC(C)(C)C]

wherein convenient reaction conditions are disclosed hereinbefore.

The compound of formula XIX is conveniently prepared using the process disclosed in WO 2004/016601 (preparation 9, page 23).

The above route is conveniently illustrated in Scheme 5

### Route 6

According to a further aspect of the invention we provide a process for the preparation of the compound of formula II which process comprises reacting the compound of formula XXVII

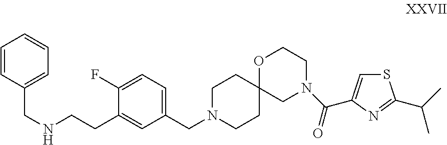 [SMILES: CC(C)c1nc(C(=O)N2CCOC3(CCN(Cc4ccc(F)c(CCNCc5ccccc5)c4)CC3)C2)cs1]

with the compound of formula XIV

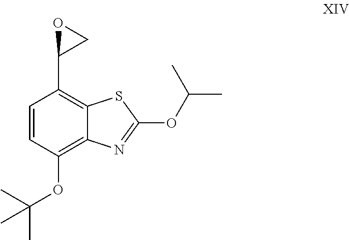 [SMILES: CC(C)Oc1nc2c(OC(C)(C)C)ccc([C@@H]3CO3)c2s1]

in a suitable solvent for example N,N-dimethylformamide, N,N-dimethylacetamide, dimethylsulfoxide or 4-methyl-2-pentanol; at a temperature, for example in the range 20 to 150° C. to give the compound of formula XXV

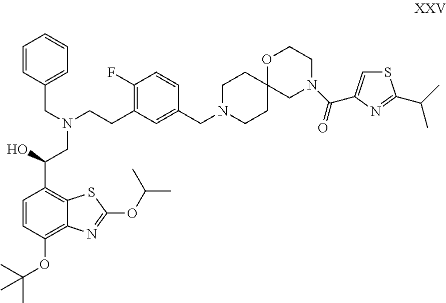 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1]

followed by deprotection, wherein convenient reaction conditions are disclosed hereinbefore, to give a compound of formula II.

We have found that simple benzothiazolones of the type XIV require protecting groups (O,O′ or O, N) to increase stability allowing isolation and subsequent chemical manipulation. We have unexpectedly found that the specific combination of t-butyl and isopropyl groups as shown, is stable enough to allow the chemistry used in formation of the parent benzothiazolone and epoxide derivative; the subsequent epoxide opening can be achieved and these specific protecting groups can be easily removed to allow formation of the compound of formula II or its salt I.

Moreover we have found that the use of a benzyl protecting group on the amine of formula XX produces a cleaner coupling reaction giving a higher yield and/or higher purity product of formula XXV. Despite the addition of benzylation and debenzylation stages, the overall yield and ease of isolation of the compound of formula II or its salt may be advantageous over the process outlined in Scheme 2 hereinbefore.

The compound of formula XXVII is conveniently prepared from the compound of formula XX or any other suitable alternate salt there of (or the neutral, parent amine)

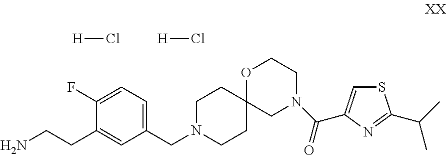 [SMILES: Cl ; Cl ; CC(C)c1nc(C(=O)N2CCOC3(CCN(Cc4ccc(F)c(CCN)c4)CC3)C2)cs1]

wherein convenient reaction conditions are disclosed hereinbefore.

The compound of formula XX is conveniently prepared from the compound of formula XXI

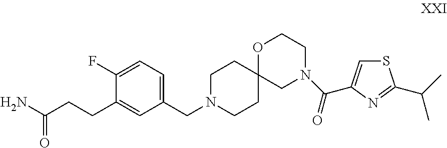 [SMILES: CC(C)c1nc(C(=O)N2CCOC3(CCN(Cc4ccc(F)c(CCC(N)=O)c4)CC3)C2)cs1]

wherein convenient reaction conditions are disclosed hereinbefore.

The compound of formula XXI is conveniently prepared from the compound of formula XXII

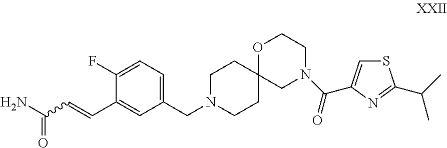 [SMILES: CC(C)c1nc(C(=O)N2CCOC3(CCN(Cc4ccc(F)c(C=CC(N)=O)c4)CC3)C2)cs1]

wherein convenient reaction conditions are disclosed hereinbefore.

The compound of formula XXII is conveniently prepared from the compound of formula XXIII

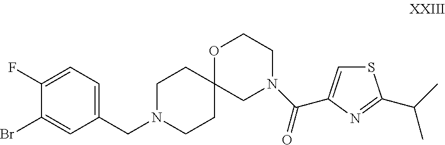 [SMILES: CC(C)c1nc(C(=O)N2CCOC3(CCN(Cc4ccc(F)c(Br)c4)CC3)C2)cs1]

wherein convenient reaction conditions are disclosed hereinbefore.

The compound of formula XXIII is conveniently prepared by reaction of the compound of formula IX

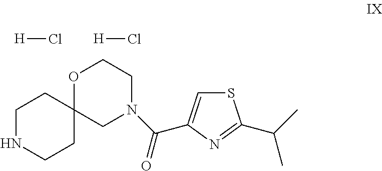

with the compound of formula XXIV

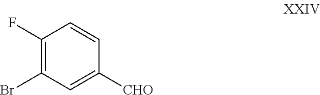

wherein convenient reaction conditions are disclosed hereinbefore.

The compound of formula IX is prepared by reaction of the compound of formula X

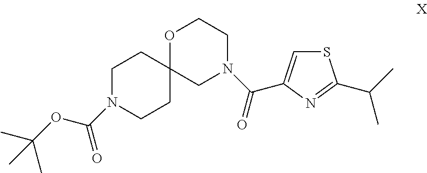 [SMILES: CC(C)c1nc(C(=O)N2CCOC3(CCN(C(=O)OC(C)(C)C)CC3)C2)cs1]

wherein convenient reaction conditions are disclosed hereinbefore

The compound of formula X is prepared by reaction of the compound of formula XI

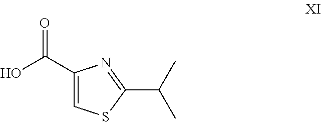

and the compound of formula XII or any other suitable alternate salt there of

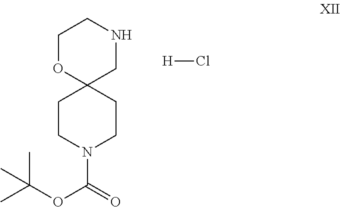 [SMILES: CC(C)(C)OC(=O)N1CCC2(CC1)CNCCO2 ; Cl]

wherein convenient reaction conditions are disclosed hereinbefore

The compound of formula XI may be obtained using the process set out in WO-1999/038862 (page 37, preparation 4).

The compound of formula XII may be obtained from WuXi Pharma Tech.

The compound of formula XXIV may be obtained from Sigma Aldrich.

The compound of formula XIV is conveniently prepared in-situ or isolated from the compound of formula XV

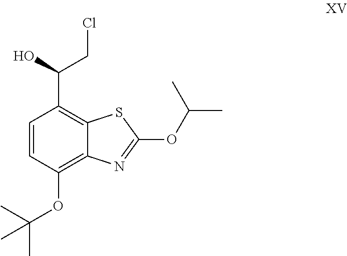 [SMILES: CC(C)Oc1nc2c(OC(C)(C)C)ccc([C@@H](O)CCl)c2s1]

wherein convenient reaction conditions are disclosed hereinbefore

The compound of formula XV may conveniently be prepared from the compound of formula XVI

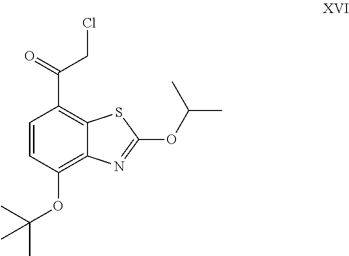 [SMILES: CC(C)Oc1nc2c(OC(C)(C)C)ccc(C(=O)CCl)c2s1]

wherein convenient reaction conditions are disclosed hereinbefore

The compound of formula XVI may conveniently be prepared from the compound of formula XVII

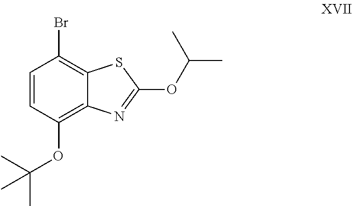 [SMILES: CC(C)Oc1nc2c(OC(C)(C)C)ccc(Br)c2s1]

wherein convenient reaction conditions are disclosed hereinbefore

or it may be obtained directly from the compound of formula XIX

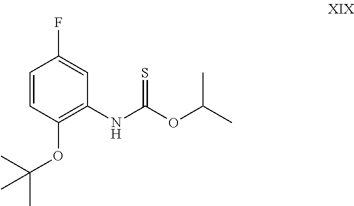 [SMILES: CC(C)OC(=S)Nc1cc(F)ccc1OC(C)(C)C]

wherein convenient reaction conditions are disclosed hereinbefore

The compound of formula XVII may conveniently be prepared from the compound of formula XVIII

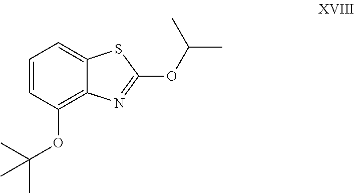 [SMILES: CC(C)Oc1nc2c(OC(C)(C)C)cccc2s1]

wherein convenient reaction conditions are disclosed hereinbefore

The compound of formula XVIII is conveniently prepared from the compound of formula XIX

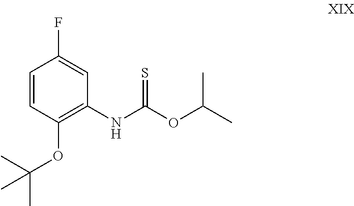 [SMILES: CC(C)OC(=S)Nc1cc(F)ccc1OC(C)(C)C]

wherein convenient reaction conditions are disclosed hereinbefore

The compound of formula XIX is conveniently prepared using the process disclosed in WO 2004/016601 (preparation 9, page 23).

The above route is conveniently illustrated in Scheme 6

### Route 7

In a further aspect of the invention we provide a process for the preparation of the compound of formula II

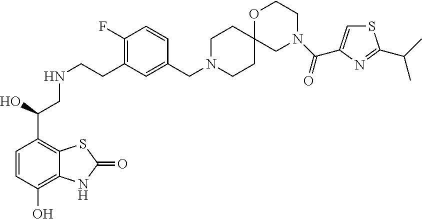 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

which comprises reaction of the compound of formula III or any other suitable alternate salt there of

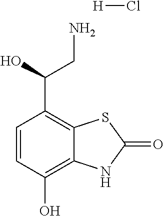

and the compound of formula V

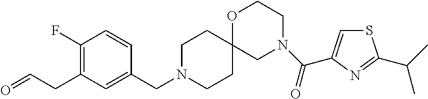 [SMILES: CC(C)c1nc(C(=O)N2CCOC3(CCN(Cc4ccc(F)c(CC=O)c4)CC3)C2)cs1]

wherein convenient reaction conditions are disclosed hereinbefore

The compound III may be conveniently prepared from compound XV

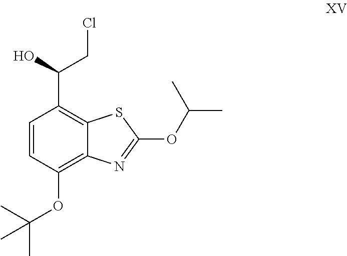 [SMILES: CC(C)Oc1nc2c(OC(C)(C)C)ccc([C@@H](O)CCl)c2s1]

By treatment with an aminating agent e.g. sodium bis(trimethylsilyl)amide in a suitable solvent e.g. tetrahydrofuran or 2-methyltetrahydrofuran at a temperature between 5-75° C. followed by treatment with hydrochloric acid in a suitable solvent e.g. isopropyl alcohol at a temperature between 5-75° C.

The compound of formula XV may conveniently be prepared from the compound of formula XVI

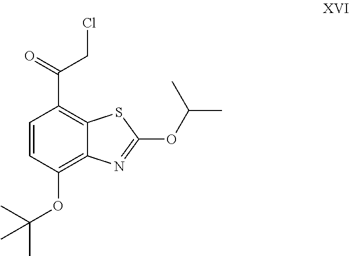 [SMILES: CC(C)Oc1nc2c(OC(C)(C)C)ccc(C(=O)CCl)c2s1]

wherein convenient reaction conditions are disclosed hereinbefore

The compound of formula XVI may conveniently be prepared from the compound of formula XVII

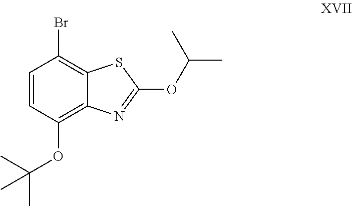 [SMILES: CC(C)Oc1nc2c(OC(C)(C)C)ccc(Br)c2s1]

wherein convenient reaction conditions are disclosed hereinbefore

or it may be obtained directly from the compound of formula XIX

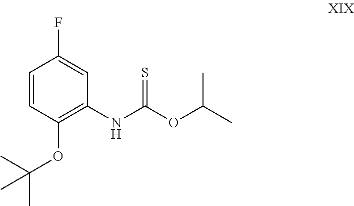 [SMILES: CC(C)OC(=S)Nc1cc(F)ccc1OC(C)(C)C]

wherein convenient reaction conditions are disclosed hereinbefore

The compound of formula XVII may conveniently be prepared from the compound of formula XVIII

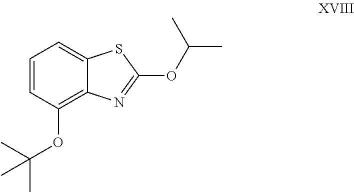 [SMILES: CC(C)Oc1nc2c(OC(C)(C)C)cccc2s1]

wherein convenient reaction conditions are disclosed hereinbefore

The compound of formula XVIII is conveniently prepared from the compound of formula XIX

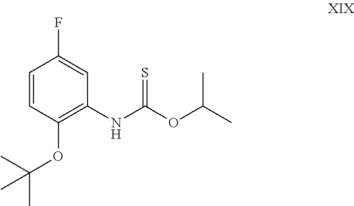 [SMILES: CC(C)OC(=S)Nc1cc(F)ccc1OC(C)(C)C]

wherein convenient reaction conditions are disclosed hereinbefore

The compound of formula XIX is conveniently prepared using the process disclosed in WO 2004/016601 (preparation 9, page 23).

The compound of formula V is conveniently prepared from the compound of formula VI or any other suitable alternate salt there of

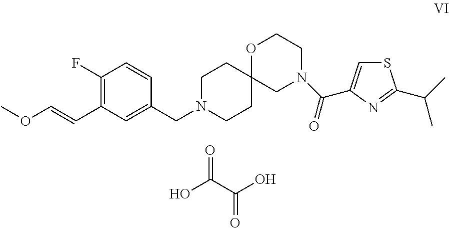

wherein convenient reaction conditions are disclosed hereinbefore

The compound of formula VI is prepared from the compound of formula VII

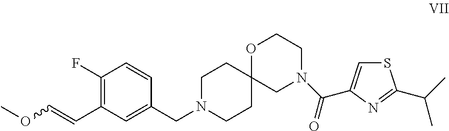 [SMILES: COC=Cc1cc(CN2CCC3(CC2)CN(C(=O)c2csc(C(C)C)n2)CCO3)ccc1F]

wherein convenient reaction conditions are disclosed hereinbefore

The compound of formula VII is prepared by reaction of the compound of formula VIII

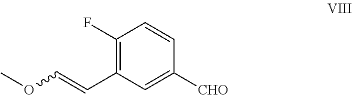

or compound VIIIa

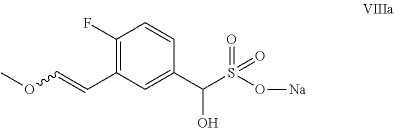

with the compound of formula IX or any other suitable alternate salt there of

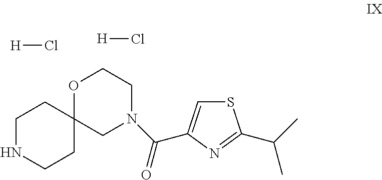

wherein convenient reaction conditions are disclosed hereinbefore

The compound of formula VIII is conveniently prepared using the method disclosed in WO 2009/098448 in Example 47E on page 202.

The compound of formula IX is prepared by reaction of the compound of formula X

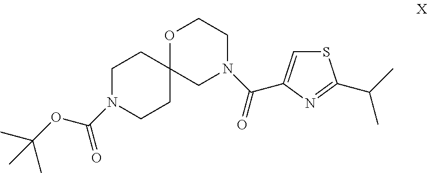 [SMILES: CC(C)c1nc(C(=O)N2CCOC3(CCN(C(=O)OC(C)(C)C)CC3)C2)cs1]

wherein convenient reaction conditions are disclosed hereinbefore

The compound of formula X is prepared by reaction of the compound of formula XI

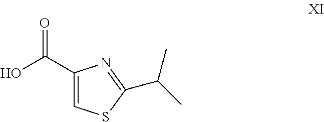

and the compound of formula XII or any other suitable alternate salt there of

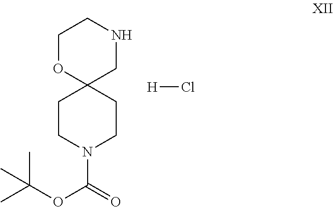

wherein convenient reaction conditions are disclosed hereinbefore

The compound of formula XI may be obtained using the process set out in WO-1999/038862 (page 37, preparation 4).

The compound of formula XII may be obtained from WuXi Pharma Tech.

The above route is conveniently illustrated in Scheme 7

According to a further aspect of the invention we provide a process for the preparation of a compound of formula IIa or IIb as set out below and pharmaceutically acceptable salts thereof,

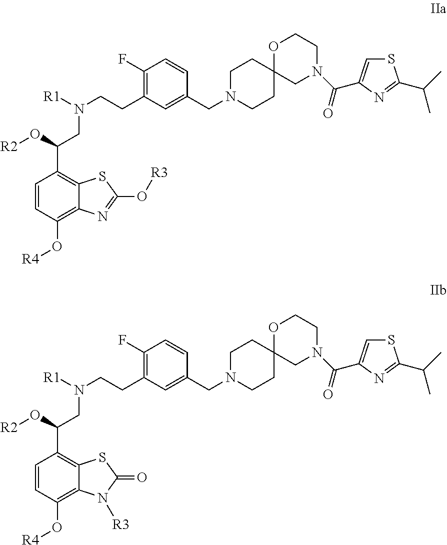

wherein R1 represents a suitable protecting group for example benzyl, tosyl, nosyl, BOC, TMS, FMOC.

wherein R2 represents a suitable protecting group for example benzyl, BOC, trimethylsilyl, triisopropylsilyl, tert-butyldimethylsilyl or tert-butyldiphenylsilyl.

wherein R3 and R4 represents a suitable protecting group for example ethyl, isopropyl, t-butyl, allyl, prenyl, benzyl, trisopropyl silyl, tert-butyl dimethyl silyl or tert-butyl diphenylsilyl,

using any one of routes 1-6 disclosed above and using intermediate products comprising groups R1, R2, R3 and R4 as appropriate.

In a further aspect the compound of formula II is converted into a pharmaceutically acceptable salt such as its dicamsylate or fumarate, directly from the solution it was formed in by the addition of a suitable acid, for example by use of a methyl tetrahydrofuran solution of II as described previously and treatment with camphoric sulfonic acid.

### Intermediates

The following intermediate compounds are novel and each represents a separate and independent aspect of the invention.

TABLE 1

| Structure | Name | Formula |
|---|---|---|
| 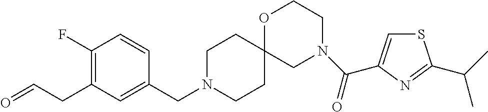 [SMILES: CC(C)c1nc(C(=O)N2CCOC3(CCN(Cc4ccc(F)c(CC=O)c4)CC3)C2)cs1] | 2-[2-fluoro-5-[[4-(2-isopropylthiazole-4- carbonyl)-1-oxa-4,9- diazaspiro[5.5]undecan-9- yl]methyl]phenyl]acetaldehyde | V |
| 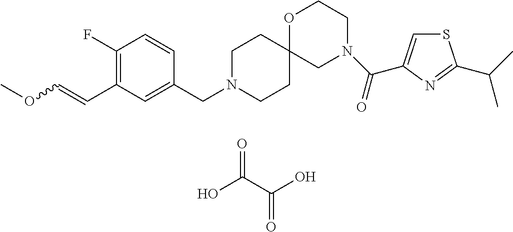 | [3-[[4-fluoro-3-[2- methoxyvinyl]phenyl]methyl]-7-oxa-3,10- diazaspiro[5.5]undecdan-10-yl]-(2- isopropylthiazol-4-yl)methanone; oxalic acid; | VI |
| 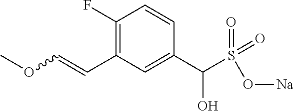 | [[4-fluoro-3-[(E)-2-methoxyvinyl]phenyl]- hydroxy-methyl]sulfonyloxysodium | VIIIa |
| 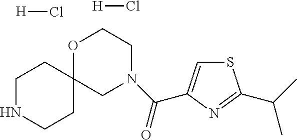 | (2-isopropylthiazol-4-yl)-(1-oxa-4,9- diazaspiro[5.5]undecan-4-yl)methanone dihydrochloride | IX |
| 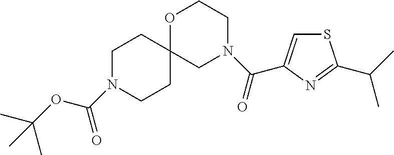 [SMILES: CC(C)c1nc(C(=O)N2CCOC3(CCN(C(=O)OC(C)(C)C)CC3)C2)cs1] | tert-butyl 4-(2-isopropylthiazole-4- carbonyl)-1-oxa-4,9- diazaspiro[5.5]undecane-9-carboxylate | X |
| 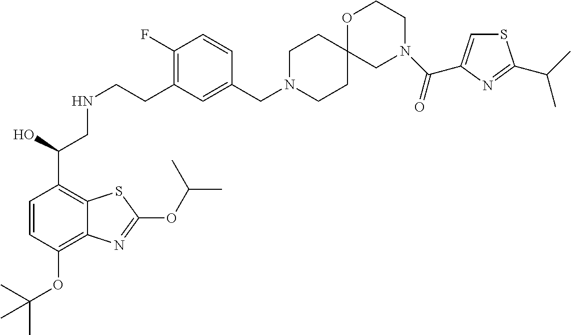 [SMILES: CC(C)Oc1nc2c(OC(C)(C)C)ccc([C@@H](O)CNCCc3cc(CN4CCC5(CC4)CN(C(=O)c4csc(C(C)C)n4)CCO5)ccc3F)c2s1] | [9-[[3-[2-[[(2R)-2-(4-tert-butoxy-2- isopropoxy-1,3-benzothiazol-7-yl)-2- hydroxy-ethyl]amino]ethyl]-4-fluoro- phenyl]methyl]-1-oxa-4,9- diazaspiro[5.5]undecan-4-yl]-(2- isopropylthiazol-4-yl)methanone | XIII |
| 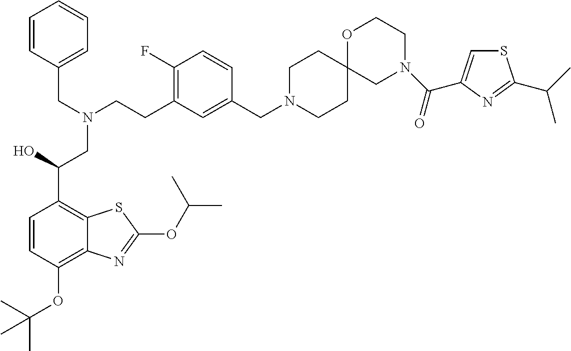 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1] | [9-[[3-[2-[benzyl-[(2R)-2-(4-tert-butoxy-2- isopropoxy-1,3-benzothiazol-7-yl)-2- hydroxy-ethyl]amino]ethyl]-4-fluoro- phenyl]methyl]-1-oxa-4,9- diazaspiro[5.5]undecan-4-yl]-(2- isopropylthiazol-4-yl)methanone | XXV |
| 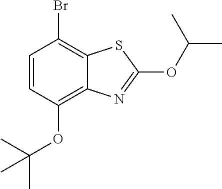 [SMILES: CC(C)Oc1nc2c(OC(C)(C)C)ccc(Br)c2s1] | 7-bromo-4-tert-butoxy-2-isopropoxy-1,3- benzothiazole | XVII |
| 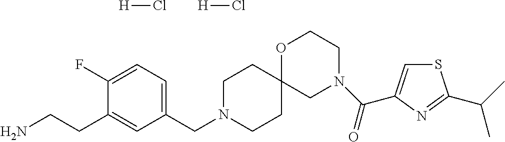 | [9-[[3-(2-aminoethyl)-4-fluoro- phenyl]methyl]-1-oxa-4,9- diazaspiro[5.5]undecan-4-yl]-(2- isopropylthiazol-4-yl)methanone dihydrochloride | XX |
| 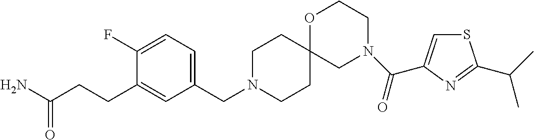 [SMILES: CC(C)c1nc(C(=O)N2CCOC3(CCN(Cc4ccc(F)c(CCC(N)=O)c4)CC3)C2)cs1] | 3-[2-fluoro-5-[[4-(2-isopropylthiazole-4- carbonyl)-1-oxa-4,9- diazaspiro[5.5]undecan-9- yl]methyl]phenyl]propanamide | XXI |
| 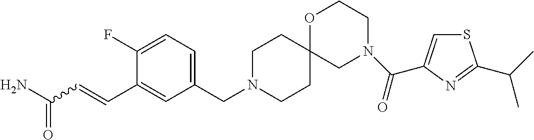 [SMILES: CC(C)c1nc(C(=O)N2CCOC3(CCN(Cc4ccc(F)c(C=CC(N)=O)c4)CC3)C2)cs1] | 3-[2-fluoro-5-[[4-(2-isopropylthiazole-4- carbonyl)-1-oxa-4,9- diazaspiro[5.5]undecan-9- yl]methyl]phenyl]prop-2-enamide | XXII |
| 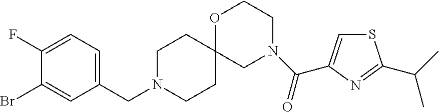 [SMILES: CC(C)c1nc(C(=O)N2CCOC3(CCN(Cc4ccc(F)c(Br)c4)CC3)C2)cs1] | [9-[(3-bromo-4-fluoro-phenyl)methyl]-1- oxa-4,9-diazaspiro[5.5]undecan-4-yl]-(2- isopropylthiazol-4-yl)methanone | XXIII |
| 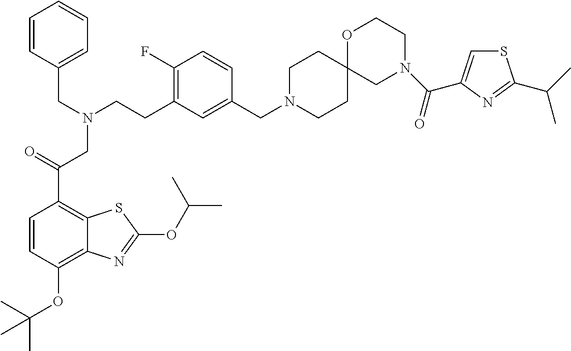 [SMILES: CC(C)Oc1nc2c(OC(C)(C)C)ccc(C(=O)CN(CCc3cc(CN4CCC5(CC4)CN(C(=O)c4csc(C(C)C)n4)CCO5)ccc3F)Cc3ccccc3)c2s1] | 2-[benzyl-[2-[2-fluoro-5-[[4-(2- isopropylthiazole-4-carbonyl)-1-oxa-4,9- diazaspiro[5.5]undecan-9- yl]methyl]phenyl]ethyl]amino]-1-(4-tert- butoxy-2-isopropoxy-1,3-benzothiazol-7- yl)ethanone | XXVI |
| 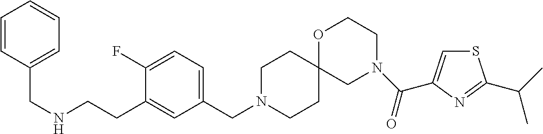 [SMILES: CC(C)c1nc(C(=O)N2CCOC3(CCN(Cc4ccc(F)c(CCNCc5ccccc5)c4)CC3)C2)cs1] | [9-[[3-[2-(benzylamino)ethyl]-4-fluoro- phenyl]methyl]-1-oxa-4,9- diazaspiro[5.5]undecan-4-yl]-(2- isopropylthiazol-4-yl)methanone | XXVII |
| 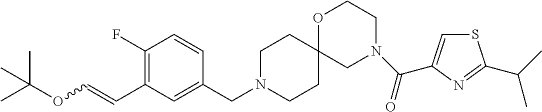 [SMILES: CC(C)c1nc(C(=O)N2CCOC3(CCN(Cc4ccc(F)c(C=COC(C)(C)C)c4)CC3)C2)cs1] | [9-[[3-[(E)-2-tert-butoxyvinyl]-4-fluoro- phenyl]methyl]-1-oxa-4,9- diazaspiro[5.5]undecan-4-yl]-(2- isopropylthiazol-4-yl)methanone | XXVIII |
| 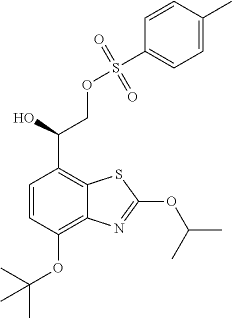 [SMILES: Cc1ccc(S(=O)(=O)OC[C@H](O)c2ccc(OC(C)(C)C)c3nc(OC(C)C)sc23)cc1] | [(2R)-2-(4-tert-butoxy-2-isopropoxy-1,3- benzothiazol-7-yl)-2-hydroxy-ethyl] 4- methylbenzenesulfonate | XXIX |
| 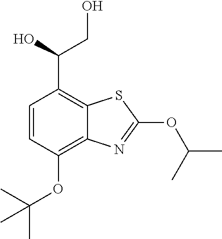 [SMILES: CC(C)Oc1nc2c(OC(C)(C)C)ccc([C@@H](O)CO)c2s1] | (1R)-1-(4-tert-butoxy-2-isopropoxy-1,3- benzothiazol-7-yl)ethane-1,2-diol | XXX |
| 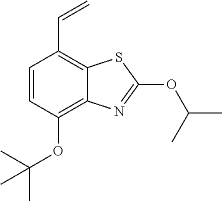 [SMILES: C=Cc1ccc(OC(C)(C)C)c2nc(OC(C)C)sc12] | 4-tert-butoxy-2-isopropoxy-7-vinyl-1,3- benzothiazole | XXXI |

The invention will now be illustrated but not limited by reference to the following specific description and Examples.

### EXAMPLE 1

### 7-[(1R)-2-[2-[2-fluoro-5-[[4-(2-isopropylthiazole-4-carbonyl)-1-oxa-4,9-diazaspiro[5.5]undecan-9-yl]methyl]phenyl]ethylamino]-1-hydroxy-ethyl]-4-hydroxy-3H-1,3-benzothiazol-2-one-di[[(1S,4R)-7,7-dimethyl-2-oxo-norbornan-1-yl]methanesulfonic acid]salt

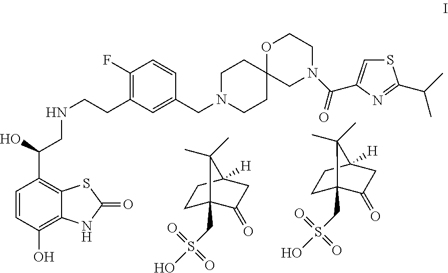

A solution of [(1S,4R)-7,7-dimethyl-2-oxo-norbornan-1-yl]methanesulfonic acid (7.80 g; 33.10 mmoles) in deionised water (1.5 mL) and isopropanol (11.4 mL) was stirred at RT for 30 minutes. A crude solution of 7-[(1R)-2-[2-[2-fluoro-5-[[4-(2-isopropylthiazole-4-carbonyl)-1-oxa-4,9-diazaspiro[5.5]undecan-9-yl]methyl]phenyl]ethylamino]-1-hydroxy-ethyl]-4-hydroxy-3H-1,3-benzothiazol-2-one II in 2-methyltetrahydrofuran (131.20 g @ 4.3% w=5.69 g; 8.50 mmoles) was then added and the mixture was stirred for 30 minutes. A seed of title compound I (7 mg) was added and the mixture was stirred at RT for at least 24 hours. The resulting solid was then collected via filtration and the filter cake was washed with isopropanol (17 mL) then dried in-vacuo at 40° C. to give title compound I as a white solid (8.58 g @ 89.9% w=7.71 g; 26.48 mmoles).

m/z 670.20 [M+H]⁺

1H NMR (500 MHz, CD₃OD) δ 8.01-7.84 (m, 1H), 7.84-7.67 (m, 1H), 7.57-7.40 (m, 1H), 7.28-7.13 (m, 1H), 7.08-6.93 (m, 1H), 6.82-6.69 (m, 1H), 5.07 (dt, J=7.9, 15.8 Hz, 1H), 4.54-4.22 (m, 2H), 4.01-3.55 (m, 6H), 3.55-3.02 (m, 13H), 2.77 (d, J=14.8 Hz, 2H), 2.67-2.52 (m, 2H), 2.39-2.27 (m, 2H), 2.27-2.10 (m, 2H), 2.09-1.69 (m, 8H), 1.62 (ddd, J=4.7, 9.3, 14.0 Hz, 2H), 1.54-1.23 (m, 8H), 1.08 (s, 6H), 0.83 (s, 6H).

### EXAMPLE 2

### 7-[(1R)-2-[2-[2-fluoro-5-[[4-(2-isopropylthiazole-4-carbonyl)-1-oxa-4,9-diazaspiro[5.5]undecan-9-yl]methyl]phenyl]ethylamino]-1-hydroxy-ethyl]-4-hydroxy-3H-1,3-benzothiazol-2-one

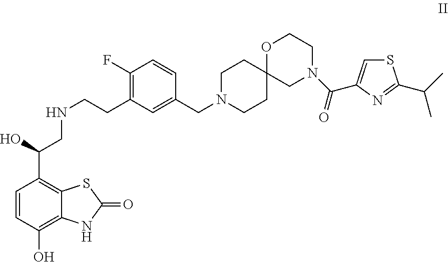 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

### a) from [3-[[4-fluoro-3-[2-methoxyvinyl]phenyl]methyl]-7-oxa-3,10-diazaspiro[5.5]undecan-10-yl]-(2-isopropylthiazol-4-yl)methanone VII

Vessel 1 was charged with hydrochloric acid (2 M; 5.5 L) and heated to 50° C. with stirring for 30 minutes. To this was added a solution of [3-[[4-fluoro-3-[2-methoxyvinyl]phenyl]methyl]-7-oxa-3,10-diazaspiro[5.5]undecan-10-yl]-(2-isopropylthiazol-4-yl)methanone VII in 2-methyltetrahydrofuran (10.47 kg @ 16.1% w=3.56 moles). The mixture was stirred at 50° C. for 2 hours then cooled to 0° C. and the phases were separated. The lower aqueous phase was basified with aqueous sodium bicarbonate (8.0% w; 7.9 L) and extracted into 2-methyltetrahydrofuran (6.6 L). The upper phase was collected, dried (sodium sulphate) and stored at −18° C. A separate hydrogenation vessel (Vessel 2) was charged with 7-[(1R)-2-amino-1-hydroxy-ethyl]-4-hydroxy-3H-1,3-benzothiazol-2-one hydrochloride III (0.65 Kg; 2.87 moles), 5% iridium on calcium carbonate (0.59 Kg), sodium sulphate (1.05 Kg) & N-methylpyrrolidone (8.9 L). The mixture was stirred for 20 minutes at RT before the solution prepared previously (Vessel 1) was added. The resulting mixture was heated to 50° C. under an atmosphere of 4.5 barg hydrogen with agitation for 21 hours. The mixture was then cooled to RT and filtered. To the resulting filtrate was charged 2-methyltetrahydrofuran (9.8 L) followed by aqueous citric acid solution (0.5% w; 47.2 L). The mixture was cooled to 5° C. and stirred for 20 minutes before being filtered. To the filtrate was added a further portion of 2-methyltetrahydrofuran (9.8 L) and the mixture was basified with aqueous potassium carbonate solution (18% w; 2.8 L) then the upper organic phase was collected. The lower aqueous phase was then extracted twice with 2-methyltetrahydrofuran (9.9 L and 4.9 L). All organic phases were combined and washed with aqueous sodium chloride solution (20% w; 2.3 L) to afford a solution of title compound II in 2-methyltetrahydrofuran, (8.56 kg @ 12.5% w=1.07 kg; 1.59 moles).

### b) from [3-[[4-fluoro-3-[2-methoxyvinyl]phenyl]methyl]-7-oxa-3,10-diazaspiro[5.5]undecan-10-yl]-(2-isopropylthiazol-4-yl)methanone; oxalic acid VI

Vessel 1 was charged with [3-[[4-fluoro-3-[2-methoxyvinyl]phenyl]methyl]-7-oxa-3,10-diazaspiro[5.5]undecan-10-yl]-(2-isopropylthiazol-4-yl)methanone; oxalic acid VI (28.70 g; 45.44 mmoles) and aqueous HCl (2 M; 73 ml). The mixture was heated to 40° C. and stirred for 2 hours. The mixture was cooled to 10° C. and basified with aqueous potassium carbonate (30% w; 70 mL) then extracted with 2-methyltetrahydrofuran (109 ml). The lower aqueous phase was separated and re-extracted with 2-methyltetrahydrofuran (109 ml). The organic phases were combined and stored. Vessel 2 (hydrogenation vessel) was charged with 7-[(1R)-2-amino-1-hydroxy-ethyl]-4-hydroxy-3H-1,3-benzothiazol-2-one hydrochloride III (10.00 g; 36.35 mmoles); 5% iridium on calcium carbonate (7.00 g) and N-methylpyrrolidone (129 ml). This mixture was stirred for 20 minutes at RT before the solution from vessel 1 was added. The mixture was heated at 65° C. at 3.9 barg with agitation for 22-36 hours. The reaction was cooled to RT and filtered; the filter cake was washed with a mixture of 2-methyltetrahydrofuran & N-methylpyrrolidinone (4:1 by volume; 53 mL). The resulting filtrate was treated with aqueous citric acid (0.85% w; 669 mL) at 15-20° C. and stirred for 30 minutes. The resulting slurry was filtered and the filter cake was washed with 2-methyltetrahydrofuran (19 mL). The resulting filtrate was then partitioned between 2-methyltetrahydrofuran (143 mL) and aqueous potassium carbonate (2 M; 334 mL) and stirred at RT for 10 minutes. The lower aqueous phase was removed and extracted twice with 2-methyltetrahydrofuran (2×143 mL). The combined organic phases were washed with aqueous brine (20% w; 72 mL) then concentrated in-vacuo at 30-35° C. to give a solution of title compound II in 2-methyltetrahydrofuran (298.0 g @ 5.41% w=16.12 g; 24.07 mmoles).

m/z C₃₃H₄₁FN₅O₅S₂ [M+H]⁺ calculated 670.2528 found 670.2540

1H NMR (500 MHz, CD₃OD) δ 7.92-7.67 (m, 1H), 7.25-7.01 (m, 2H), 7.01-6.87 (m, 1H), 6.82 (d, J=8.3 Hz, 1H), 6.62 (d, J=8.3 Hz, 1H), 4.71 (dd, J=4.1, 8.5 Hz, 1H), 3.97-3.32 (m, 8H), 3.32-3.20 (m, 1H), 3.02-2.62 (m, 6H), 2.61-2.17 (m, 4H), 1.89-1.40 (m, 4H), 1.33 (d, J=6.8 Hz, 6H).

### EXAMPLE 3

### [3-[[4-fluoro-3-[2-methoxyvinyl]phenyl]methyl]-7-oxa-3,10-diazaspiro[5.5]undecan-10-yl]-(2-isopropylthiazol-4-yl)methanone; oxalic acid

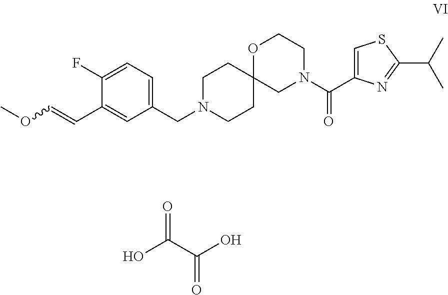 [SMILES: COC=Cc1cc(CN2CCC3(CC2)CN(C(=O)c2csc(C(C)C)n2)CCO3)ccc1F ; O=C(O)C(=O)O]

A solution of [3-[[4-fluoro-3-[2-methoxyvinyl]phenyl]methyl]-7-oxa-3,10-diazaspiro[5.5]undecan-10-yl]-(2-isopropylthiazol-4-yl)methanone VII (412 g; 0.87 moles) in 2-methyltetrahydrofuran (2.5 L) was stirred and heated to 50° C. To this was added a solution of oxalic acid (94.3 g; 1.05 moles) in 2-methyltetrahydrofuran (1.5 L) keeping the temperature of the stirred mixture at 50° C. Seed of title compound VI (0.04 g) was then added to the mixture and the solution then cooled to 5° C. over 2 hours. After stirring overnight at 5° C. the solid was filtered and washed with 2-methyltetrahydrofuran (0.8 L). The solid was then allowed to dry under vacuum at 50° C. to constant weight to give title compound VI (mixture of E &Z isomers) as a white solid (503 g; 0.81 moles).

m/z 474 [M+H]⁺

¹H NMR (400 MHz, CD₃OD) δ 8.18* (s, 1H), 7.89 (s, 1H), 7.51^{†} (d, J=5.9 Hz, 1H), 7.25 (d, J=13.1 Hz, 2H), 7.14-6.98 (m, 1H), 6.37* (d, J=7.2 Hz, 1H), 6.25^{†} (s, 1H), 5.84^{†} (d, J=13.0 Hz, 1H), 5.38* (d, J=7.1 Hz, 1H), 4.05-3.47 (m, 11H), 3.47-3.01 (m, 9H), 2.34-2.00 (m, 3H), 1.99-1.64 (m, 3H), 1.37 (d, J=6.8 Hz, 8H), 1.19* (t, J=8.1 Hz, 1H).

^{†}Major isomer; *Minor isomer

### EXAMPLE 4

### [3-[[4-fluoro-3-[2-methoxyvinyl]phenyl]methyl]-7-oxa-3,10-diazaspiro[5.5]undecan-10-yl]-(2-isopropylthiazol-4-yl)methanone

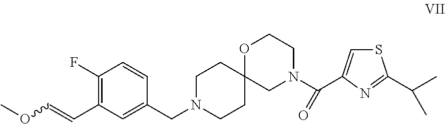 [SMILES: COC=Cc1cc(CN2CCC3(CC2)CN(C(=O)c2csc(C(C)C)n2)CCO3)ccc1F]

### a) from 4-fluoro-3-[2-methoxyvinyl]benzaldehyde VIII

(2-isopropylthiazol-4-yl)-(1-oxa-4,9-diazaspiro[5.5]undecan-4-yl)methanone dihydrochloride IX (2.50 Kg, 6.46 moles) was slurried in 2-methyltetrahydrofuran (15.1 L) at RT and treated with aqueous sodium hydroxide (5 M; 5.0 L). The bi-phasic mixture was stirred for 20 minutes and both the aqueous and organic layers were separated and retained. The aqueous layer was stirred with 2-methyltetrahydrofuran (17.5 L) for 20 minutes and the aqueous layer was separated and discarded. The organic extracts were combined and distilled at atmospheric pressure to low volume affording quantitative yield of (2-isopropylthiazol-4-yl)-(1-oxa-4,9-diazaspiro[5.5]undecan-4-yl)methanone as a 2-methyltetrahydrofuran solution (1.99 kg; 6.46 moles). This was combined with a mixture of 4-fluoro-3-[2-methoxyvinyl]benzaldehyde VIII in toluene (1.28 kg; 7.11 moles) and stirred for at least 20 minutes before being added over 3-4 hours to a slurry of sodium triacetoxyborohydride (4.23 Kg; 19.97 moles) in toluene (22.2 L) at RT. The resulting mixture was stirred at RT for 12 hours.

The mixture was quenched and diluted cautiously with aqueous acetic acid (50% w; 12.5 L) at RT. The biphasic mixture was stirred for 20 minutes and the aqueous layer separated and retained (<5° C.). The reaction mixture was further washed with aqueous acetic acid (50% w; 3×12.5 L), on each occasion retaining and combining the acidic aqueous extracts. The combined acidic aqueous extracts were then diluted with 2-methyltetrahydrofuran (12.1 L) and the mixture basified with aqueous sodium hydroxide solution (10 M; 39.0 L) at RT until pH>8.5 was reached. The resulting biphasic mixture was warmed to 33° C. and stirred for 15 minutes before the lower aqueous phase was separated and discarded. The remaining organic layer contained a 2:3 mixture of cis and trans isomers of title compound VII as a solution in 2-methyltetrahydrofuran (15.19 kg @ 18.0% w=2.73 kg; 5.76 moles).

### b) from [[4-fluoro-3-[(E)-2-methoxyvinyl]phenyl]-hydroxy-methyl]sulfonyloxysodium VIIIa

In vessel 1, a slurry of (2-isopropylthiazol-4-yl)-(1-oxa-4,9-diazaspiro[5.5]undecan-4-yl)methanone dihydrochloride IX (16.3 kg; 42.6 moles) in 2-methyltetrahydrofuran (97.0 kg) was stirred at <30° C. for 30 minutes before being treated with aqueous sodium hydroxide (5 M, 35.9 kg) and stirred for a further 30 minutes. The resulting biphasic mixture was separated and the lower, aqueous phase was extracted with 2-methyltetrahydrofuran (43.0 kg). The combined organic phases were then concentrated in-vacuo until a still-head temperature of 77-78° C. was reached and the water content of the concentrated solution was less than 1.0% w (Karl Fischer). Vessel 2 was charged with [[4-fluoro-3-[(E)-2-methoxyvinyl]phenyl]-hydroxy-methyl]sulfonyloxysodium VIIIa (13.3 kg; 47.0 moles) and toluene (127.1 kg) followed by aqueous sodium bicarbonate (11% w; 308.1 kg). The resulting biphase was stirred at 15-20° C. for 30 minutes until all material was dissolved. The phases were then separated and the lower, aqueous phase was extracted with toluene (60.1 kg). The organic phases were then combined and washed with aqueous brine (29% w; 84.4 kg). The contents of vessel 2 were then added to vessel 1 with stirring over 30 minutes maintaining a temperature of 15-20° C. Vessel 3 was charged with sodium triacetoxyborohydride (27.1 kg; 128.0 moles) and toluene (127.1 kg) and stirred for 30 minutes at <20° C. The contents of vessel 1 were then added to vessel 3 with stirring over a period of at least 1 hour maintaining a temperature of 15-20° C. The resulting mixture was then stirred at 15-20° C. for 16 hours. The mixture was then cooled to 0-5° C. and quenched with aqueous acetic acid (50% w; 86.9 kg) with stirring over a period of at least 45 minutes maintaining a temperature <25° C. The lower aqueous phase was removed and the organic phase was extracted with aqueous acetic acid (50% w; 5×86.9 kg). The combined aqueous phases were then stirred with deionised water (86.4 kg) and 2-methyltetrahydrofuran (70.1 kg) for 30 minutes at 15-20° C. The pH of the aqueous phase was adjusted to 7.8-8.5 using aqueous sodium hydroxide (40% w; 78.2 kg) and the mixture was heated to 30-35° C. and stirred for 30 minutes. The lower, aqueous phase was removed and the organic layer was assayed (HPLC) for title compound VII (18.7 kg @ 100% w; 39.5 moles).

¹H NMR (400 MHz, DMSO) δ 8.00 (s, 2H), 7.83 (s, 1H), 7.47-6.90 (m, 7H), 6.42* (d, J=7.1 Hz, 1H), 5.81^{†} (d, J=13.1 Hz, 1H), 5.31* (d, J=6.9 Hz, 1H), 4.05-2.99 (m, 27H), 2.82-2.02 (m, 31H), 2.00-0.95 (m, 24H).

^{†}Major isomer; *Minor isomer

### EXAMPLE 5

### (2-isopropylthiazol-4-yl)-(1-oxa-4,9-diazaspiro[5.5]undecan-4-yl)methanone dihydrochloride

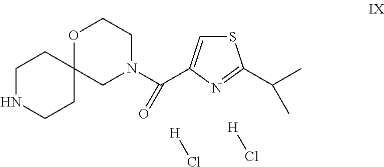

A vessel was charged with tert-Butyl-7-oxa-3,10-diazaspiro[5.5]undecane-3-carboxylate hydrochloride XII (4.00 Kg, 13.66 moles), 2-isopropylthiazole-4-carboxylic acid (2.41 Kg, 14.08 moles) and 2-methyltetrahydrofuran (28.0 L). The mixture was stirred at 5° C. and triethylamine (6.9 L, 68.19 moles) was added. Next, 2-propanephosphonic acid anhydride (T3P) in tetrahydrofuran (1.62 M; 10.9 L, 17.66 moles) was added and the reaction was warmed to RT and stirred for 1 hour. Water (28.0 L) was added and the layers were separated. The organic layer was retained and washed with water (16.0 L). The organic layer was then concentrated at 30° C. under vacuum down to ˜20 L & diluted with isopropyl alcohol (16.0 L). This concentration/dilution cycle was then repeated and a final distillation at 30° C. under vacuum gave a solution of tert-butyl-4-(2-isopropylthiazole-4-carbonyl)-1-oxa-4,9-diazaspiro[5.5]undecane-9-carboxylate X in isopropyl alcohol and 2-methyltetrahydrofuran (˜20:1). A solution of HCl in isopropyl alcohol (5-6 N, 16.2 L; 89.00 moles) was then added and the reaction heated at 40° C. for 3 hours. The reaction was then cooled to RT and methyl tert-butyl ether (8.0 L) was added to the vessel over a period of 1 hour; the resulting mixture was stirred for 24 hours. The precipitated solid was collected by filtration and washed with methyl tert-butyl ether (8.0 L). The solid was then dried at 50° C. under vacuum to constant weight giving title compound IX as a white solid (4.61 Kg; 12.05 moles).

m/z C₁₅H₂₄N₃O₂S [M+H]⁺ calculated 310.1589 found 310.1583

¹H NMR (400 MHz, d₆-DMSO) δ 9.2-8.95 (m, 2H), 8.05 (s, 1H), 3.85-3.5 (m, 6H), 3.32 (m, 1H), 3.15-3.0 (m, 2H), 3.0-2.85 (m, 2H), 2.0-1.90 (m, 2H), 1.85-1.60 (m, 2H), 1.34 (d, J=6.4 Hz, 6H).

### EXAMPLE 6

### 7-[(1R)-2-[2-[2-fluoro-5-[[4-(2-isopropylthiazole-4-carbonyl)-1-oxa-4,9-diazaspiro[5.5]undecan-9-yl]methyl]phenyl]ethylamino]-1-hydroxy-ethyl]-4-hydroxy-3H-1,3-benzothiazol-2-one

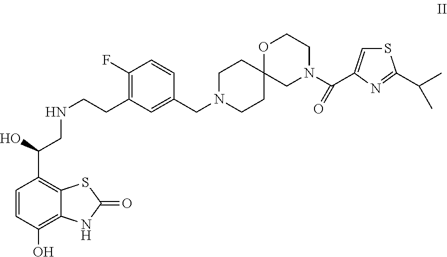 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]

### a) from [9-[[3-[2-[[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone XIII

A solution of [9-[[3-[2-[[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone XIII (7.00 g; 9.11 mmoles) in 2-methyltetrahydrofuran (12 mL) was treated with aqueous HCl (5 M; 40 mL; 200.00 mmoles) and the resulting mixture was stirred at RT for 16 hours. After addition of further 2-methyltetrahydrofuran (25 mL), the mixture was basified to pH˜14 using aqueous NaOH (10 M). The resulting biphase was separated and the lower aqueous phase was washed with 2-methyltetrahydrofuran (25 mL). The aqueous phase was acidified to pH˜8 using aqueous HCl (5 M) and extracted with 2-methyltetrahydrofuran (2×25 mL). The combined organic extracts were then dried over magnesium sulfate, filtered and concentrated in-vacuo to give a solution of title compound II in 2-methyltetrahydrofuran (21.17 g @ 18.09% w=3.83 g; 5.71 mmoles).

### b) From [9-[[3-[2-[benzyl-[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone XXV

A solution of [9-[[3-[2-[benzyl-[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone XXV (100 mg, 0.12 mmol) in formic acid (2 mL) was treated with palladium black (100 mg, 100% w) and the resulting suspension was left to stir for 16 hours. The suspension was then filtered and evaporated to give the crude product as a glass/resin. Purification by flash chromatography (DCM/MeOH/NH₃, 90/9/1) gave title compound II as a white solid (60 mg, 90 μM).

Analytical data as given in Example 2.

### EXAMPLE 7

### [9-[[3-[2-[[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone

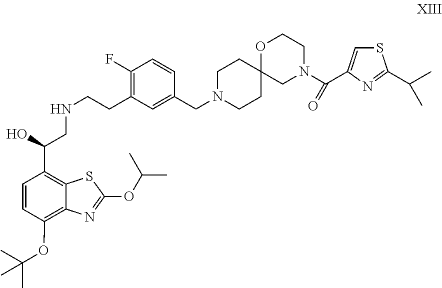 [SMILES: CC(C)Oc1nc2c(OC(C)(C)C)ccc([C@@H](O)CNCCc3cc(CN4CCC5(CC4)CN(C(=O)c4csc(C(C)C)n4)CCO5)ccc3F)c2s1]

In vessel 1, a mixture of (1R)-1-(4-tert-butoxy-2-isopropoxy-1,3-benzothiazol-7-yl)-2-chloro-ethanol XV (40.0 g; 105.9 mmoles) and potassium carbonate (29.6 g; 211.7 mmoles) was dissolved into dimethylacetamide (190 mL) and water (10 mL) at 55° C. and stirred for 4 hours. In vessel 2, a mixture of [9-[[3-(2-aminoethyl)-4-fluoro-phenyl]methyl]-1-oxa-4,9-diazaspiro[5.5]undecan-4-yl]-(2-isopropylthiazol-4-yl)methanone dihydrochloride salt XX (62.32 g; 116.5 mmoles), aqueous NaOH (2 M; 300 mL) and 2-methyltetrahydrofuran (300 mL) was stirred for 30 minutes. The resulting biphasic mixture was separated and the aqueous phase was extracted with 2-methyltetrahydrofuran (300 mL). The combined organic phases were evaporated to dryness in-vacuo then redissolved into dimethylacetamide (190 mL) and water (10 mL). The resulting solution was added to the contents of vessel 1 and heated to 80° C. and stirred for 16 hours. After cooling, the mixture was partitioned between methyl tert-butyl ether (600 mL) and water (600 mL); the lower, aqueous phase was then extracted twice with methyl tert-butyl ether (2×400 mL). The combined organic phases were stirred with aqueous citric acid (10% w, 400 mL) and methanol (100 mL) to give a biphasic mixture. The organic phase was then extracted twice with aqueous citric acid (10% w, 400 mL). The combined citric acid phases were basified to pH>13-14 using aqueous NaOH (10 M) and extracted with 2-methyltetrahydrofuran (3×400 mL) to give a solution of title compound XIII in 2-methyltetrahydrofuran (1177.5 g @ 4.4% w=52.0 g; 67.7 mmoles).

¹H NMR (400 MHz, d₆-DMSO, 90° C.) δ 7.89 (s, 1H), 7.20-7.13 (m, 1H), 7.13-7.07 (m, 1H), 7.01 (dd, J=9.1, 14.1 Hz, 2H), 6.91 (d, J=8.2 Hz, 1H), 5.29 (s, 1H), 4.71 (s, 1H), 3.64 (d, J=10.9 Hz, 6H), 3.39 (s, 2H), 3.30 (s, 1H), 2.80 (d, J=5.8 Hz, 4H), 2.72 (d, J=7.0 Hz, 2H), 2.34 (d, J=21.9 Hz, 4H), 1.76-1.64 (m, 2H), 1.60-1.48 (m, 2H), 1.42 (d, J=6.2 Hz, 6H), 1.39-1.29 (m, 15H).

### EXAMPLE 8

### [9-[[3-[2-[benzyl-[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone

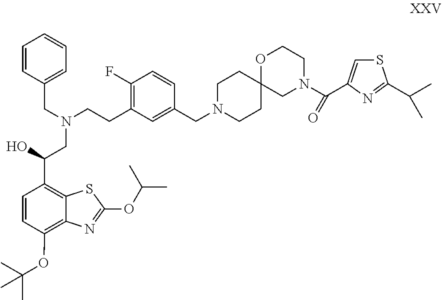 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1]

A mixture of (1R)-1-(4-tert-butoxy-2-isopropoxy-1,3-benzothiazol-7-yl)-2-chloro-ethanol XV (300 mg, 0.87 mmoles) and sodium hexamethyldisilazide (224 mg, 1.22 mmoles) were dissolved into methylisobutyl carbinol (4.5 mL) and stirred at 60° C. under nitrogen for 1 hour. A solution of [9-[[3-[2-(benzylamino)ethyl]-4-fluoro-phenyl]methyl]-1-oxa-4,9-diazaspiro[5.5]undecan-4-yl]-(2-isopropylthiazol-4-yl)-methanone XX (505 mg, 0.92 mmoles) was added and the solution was heated to 120° C. and left to stir under nitrogen for 16 hours. The mixture was cooled to RT and water (15 mL) was added and the resulting biphase was extracted with methyl tert-butyl ether (2×30 mL). The combined organics were washed with saturated brine solution (15 mL) then evaporated to dryness to give an orange oil. This material was purified by flash chromatography (2-3% MeOH in EtOAc) to give the title compound XXV as a white solid (430 mg, 0.50 mmoles).

¹H NMR (400 MHz, d₆-DMSO, 90° C.) δ 7.90 (s, 1H), 7.27-6.80 (m, 10H), 5.27 (dt, J=6.2, 12.4 Hz, 1H), 5.08 (s, 1H), 4.74 (t, J=6.2 Hz, 1H), 3.81-3.47 (m, 8H), 3.46-3.17 (m, 3H), 2.89-2.55 (m, 6H), 2.43-2.07 (m, 4H), 1.78-1.59 (m, 2H), 1.60-1.45 (m, 2H), 1.41 (d, J=6.2 Hz, 6H), 1.37-1.27 (m, 15H).

### EXAMPLE 9

### (1R)-1-(4-tert-butoxy-2-isopropoxy-1,3-benzothiazol-7-yl)-2-chloro-ethanol

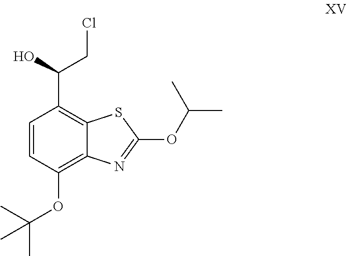 [SMILES: CC(C)Oc1nc2c(OC(C)(C)C)ccc([C@@H](O)CCl)c2s1]

A vessel was charged with 1-(4-tert-butoxy-2-isopropoxy-1,3-benzothiazol-7-yl)-2-chloro-ethanone XVI (2.00 g, 5.44 mmoles) and acetonitrile (20 mL). Pre-mixed formic acid (1.54 mL; 40.81 mmoles) and triethylamine (3.79 mL; 27.20 mmoles) complex was then added slowly to the reaction mixture and the resulting solution stirred at RT for 5 minutes. The catalyst [(S,S)-TsDpen-Ru(p-cymene)Cl] (69 mg, 0.11 mmoles) was added in a single portion and the mixture was left to stir at 20-25° C. for 2 hours. Slow addition of water (20 mL) over a period of 15 minutes caused precipitation of a light-coloured solid. After further stirring, the solid was collected via filtration; the filter cake was washed with a mixture of water and acetonitrile (2:1 by volume; 2×5 mL). The solid was dried in-vacuo @ 40° C. to give title compound XV as a pale-yellow solid (1.78 g; 5.17 mmoles).

¹H NMR (500 MHz, CDCl₃) δ=7.03 (d, J=8.2 Hz, 1H), 6.94 (d, J=8.2 Hz, 1H), 5.43 (sept., J=6.2 Hz, 1H), 4.97-4.94 (m, 1H), 3.77-3.71 (m, 2H), 2.92 (d, J=1.6 Hz, 1H), 1.43 (d, J=6.2 Hz, 6H), 1.39 (s, 9H).

### EXAMPLE 10

### 1-(4-tert-butoxy-2-isopropoxy-1,3-benzothiazol-7-yl)-2-chloro-ethanone

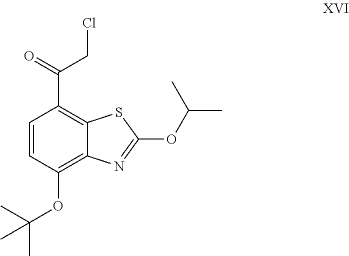 [SMILES: CC(C)Oc1nc2c(OC(C)(C)C)ccc(C(=O)CCl)c2s1]

A solution of n-butyllithium in hexanes (1.6 M, 0.41 mL, 0.65 mmoles) was added dropwise to a pre-cooled (−50° C.) solution of 7-bromo-4-tert-butoxy-2-isopropoxy-1,3-benzothiazole XVII (225 mg, 0.59 mmoles) in methyl tert-butyl ether (2.5 mL) maintaining a temperature below −45° C. The mixture was allowed to warm to −20° C. and left to stir for 30 minutes. A solution of 2-chloro-N-methoxy-N-methyl acetamide (122 mg, 0.89 mmoles) in methyl tert-butyl ether (2.5 mL) was then added dropwise maintaining a temperature below −15° C. and the mixture allowed to stir for 20 minutes. The reaction was then quenched by the addition of saturated ammonium chloride solution (2.0 mL) and water (10.0 mL). The aqueous phase was extracted with methyl tert-butyl ether (2×10 mL) and the combined organic phases were dried (MgSO₄), filtered and evaporated to give a pale orange solid. Purification by flash chromatography (isohexane/EtOAc, 95/5 to 90/10) gave title compound XVI as a beige solid (120 mg, 0.35 mmoles).

¹H NMR (500 MHz, CDCl₃) δ 7.75 (d, J=8.5 Hz, 1H), 7.11 (d, J=8.5 Hz, 1H), 5.46 (hept, J=6.2 Hz, 1H), 4.79 (s, 2H), 1.50 (s, 9H), 1.47 (d, J=6.2 Hz, 6H).

### EXAMPLE 11

### 7-bromo-4-tert-butoxy-2-isopropoxy-1,3-benzothiazole

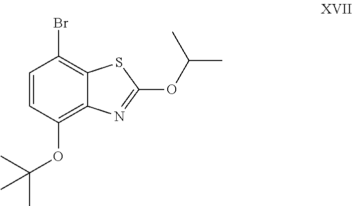 [SMILES: CC(C)Oc1nc2c(OC(C)(C)C)ccc(Br)c2s1]

To a solution of 4-tert-butoxy-2-isopropoxy-1,3-benzothiazole XVIII (13.6 g, 51.2 mmoles) in 2-methyltetrahydrofuran (300 mL) was added N-bromosuccinimide (11.0 g, 61.4 mmoles). The resulting brown solution was stirred at RT for 16 hours. Saturated brine solution (100 mL) was added and the mixture was stirred at RT for 10 minutes. The aqueous phase was separated and washed with 2-methyltetrahydrofuran (100 mL) and the combined organic phases were dried (MgSO₄), filtered and evaporated in-vacuo to give the crude, title compound XVII as a brown oil. The material was purified by column chromatography (isohexane/dichloromethane, 2:1) to give the title XVII compound as an orange oil (11.2 g, 32.6 mmoles).

¹H NMR (400 MHz, CDCl₃) δ 7.33-7.14 (m, 2H), 6.97-6.82 (m, 1H), 5.55-5.28 (m, 1H), 1.57-1.25 (m, 15H).

This compound has also been synthesised using 1,3-dibromo-5,5-dimethylhydantoin as a brominating agent under identical conditions.

### EXAMPLE 12

### 4-tert-butoxy-2-isopropoxy-1,3-benzothiazole

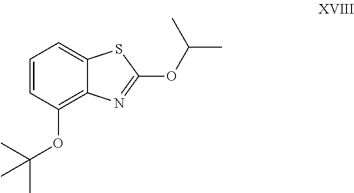 [SMILES: CC(C)Oc1nc2c(OC(C)(C)C)cccc2s1]

A solution of diisopropylamine (2.96 mL, 21.0 mmoles) in 2-methyltetrahydrofuran (10 mL) was stirred under nitrogen and cooled to −30° C. A solution of n-hexyllithium in hexanes (2.3 M, 9.14 mL, 21.0 mmoles) was then added dropwise maintaining a temperature of −25 to −30° C. The resulting mixture was stirred for 30 minutes at −30° C. A solution of O-isopropyl N-(2-tert-butoxy-5-fluoro-phenyl)carbamothioate XIX (2.00 g, 7.0 mmoles) in 2-methyltetrahydrofuran (10 mL) was then added over 60 minutes maintaining a temperature of −25 to −30° C. Once the addition was complete, the mixture was warmed to RT over 30 minutes and carefully quenched with aqueous HCl (1M; 25 mL) and stirred at RT for 10 minutes. The organic phase was then washed with saturated brine solution (25 mL), dried (MgSO₄) and evaporated in-vacuo to give the crude, title compound XVIII as a yellow-orange oil (1.81 g @ 79% w=1.43 g; 5.3 mmoles).

¹H NMR (400 MHz, CDCl₃) δ 7.35 (dd, J=0.9, 7.9 Hz, 1H), 7.10 (t, J=7.9 Hz, 1H), 7.04-6.93 (m, 1H), 5.46 (hept, J=6.2 Hz, 1H), 1.52-1.26 (m, 17H).

### EXAMPLE 13

### [9-[[3-[2-(benzylamino)ethyl]-4-fluoro-phenyl]methyl]-1-oxa-4,9-diazaspiro[5.5]undecan-4-yl]-(2-isopropylthiazol-4-yl)methanone

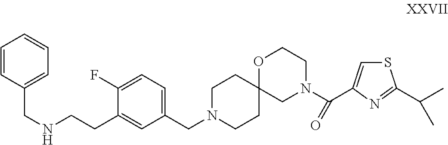 [SMILES: CC(C)c1nc(C(=O)N2CCOC3(CCN(Cc4ccc(F)c(CCNCc5ccccc5)c4)CC3)C2)cs1]

A mixture of 2-[2-fluoro-5-[[4-(2-isopropylthiazole-4-carbonyl)-1-oxa-4,9-diazaspiro[5.5]undecan-9-yl]methyl]phenyl]acetaldehyde V (11.51 g; 25.0 mmoles), benzylamine (5.47 mL; 50.0 mmoles), 5% iridium on calcium carbonate (3.45 g) and ethanol (200 mL) were charged to a hydrogenation vessel and the contents heated to 40° C. The mixture was stirred for 16 hours under a hydrogen atmosphere (4 barg). The catalyst was then filtered off and the filter cake washed with ethanol (50 mL). The filtrate was concentrated under reduced pressure and the crude mixture purified by column chromatography (2-5% methanol and 1% ammonia in dichloromethane) to give title compound XXVII as a yellow oil. (13.79 g, 19.0 mmoles)

m/z 551 [M+H]⁺

¹H NMR (400 MHz, d₆-DMSO, 90° C.): δ 7.90 (s, 1H), 7.27 (m, 4H), 7.17 (m, 2H), 7.09 (m, 1H), 6.99 (m, 1H), 3.72 (s, 2H), 3.64 (broad m, 6H), 3.39 (s, 2H), 3.31 (sep, 1H, J=6.8 Hz), 2.76 (broad m, 4H), 2.31 (broad m, 5H), 1.67 (broad m, 2H), 1.52 (broad m, 2H), 1.35 (d, J=6.8 Hz, 6H)

### EXAMPLE 14

### [9-[(3-bromo-4-fluoro-phenyl)methyl]-1-oxa-4,9-diazaspiro[5.5]undecan-4-yl]-(2-isopropylthiazol-4-yl)methanone

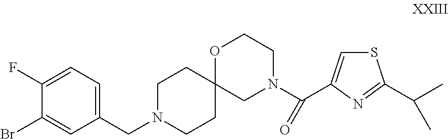 [SMILES: CC(C)c1nc(C(=O)N2CCOC3(CCN(Cc4ccc(F)c(Br)c4)CC3)C2)cs1]

To a suspension of (2-isopropylthiazol-4-yl)-(1-oxa-4,9-diazaspiro[5.5]undecan-4-yl)methanone dihydrochloride IX (140 g, 366.2 mmoles) in dichloromethane (1.68 L) at 20° C. under a nitrogen atmosphere was added triethylamine (176 mL, 1263.4 mmoles). The mixture was stirred for 1 hour, before 3-bromo-4-fluoro-benzaldehyde (78.88 g, 380.8 mmoles) was added followed by sodium triacetoxyborohydride (179.7 g, 805.5 mmoles). The reaction was then stirred at 20° C. for 18 hours. The reaction mixture was then washed with saturated sodium bicarbonate solution (3×630 mL). The organic layer was separated, dried (sodium sulphate), filtered and concentrated in-vacuo to give the title compound XXIII (211.3 g @ 80% w=169.0 g; 340.5 mmoles). This material was used in the next step without further purification.

¹H NMR (400 MHz, d₆-DMSO) δ 8.0 (s, 1H), 7.70-7.61 (m, 1H), 7.40-7.28 (m, 2H), 3.75-3.45 (m, 6H), 3.31-3.24 (m, 1H), 2.70-2.43 (m, 6H), 1.83-1.75 (m, 2H), 1.66-1.55 (m, 2H) 1.34-1.31 (d, J=6.9 Hz, 6H).

### EXAMPLE 15

3-[2-fluoro-5-[[4-(2-isopropylthiazole-4-carbonyl)-1-oxa-4,9-diazaspiro[5.5]undecan-9-yl]methyl]phenyl]prop-2-enamide

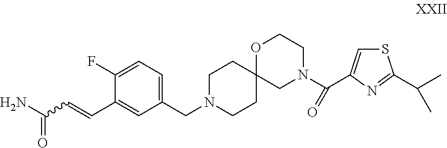 [SMILES: CC(C)c1nc(C(=O)N2CCOC3(CCN(Cc4ccc(F)c(C=CC(N)=O)c4)CC3)C2)cs1]

To a solution of [9-[(3-bromo-4-fluoro-phenyl)methyl]-1-oxa-4,9-diazaspiro[5.5]undecan-4-yl]-(2-isopropylthiazol-4-yl)methanone XXIII (211.0 g, 337.5 mmoles) in acetonitrile (1.42 L) was added acrylamide (28.8 g, 405.0 mmoles), Pd-115 (12.1 g, 16.9 mmol) and diisopropylethylamine (146.2 mL, 843.8 mmoles). The resulting mixture was heated to reflux and stirred for 16 hours. The reaction mixture was concentrated (˜400 mL) and 2-methyltetrahydrofuran (500 mL) was added. The solution was extracted with aqueous HCl (2M; 3×500 mL). The combined aqueous phases were washed with 2-methyltetrahydrofuran (205 mL). The aqueous phase was partitioned between 2-methyltetrahydrofuran (500 mL) and basified with aqueous sodium hydroxide solution (10 M, 152 mL). The organic phase was separated and the aqueous phase was extracted with 2-methyltetrahydrofuran (200 mL). The combined organic phases were concentrated in-vacuo to give the title compound XXII (211.5 g @ 73%=154.4 g; 317.3 mmoles). This material was used in the next step without further purification.

¹H NMR (400 MHz, CDCl₃) δ 7.76 (s, 1H), 7.67-7.59 (m, 1H), 7.53-7.49 (m, 1H), 7.45-7.36 (bs, 1H), 6.99-6.94 (t, J=9.84 Hz, 1H), 6.57-6.52 (d, J=15.9 Hz, 1H), 5.90-5.70 (m, 2H), 3.91-3.21 (m, 9H), 2.67-2.45 (m, 4H), 1.98-1.94 (m, 2H), 1.92-1.55 (m, 2H) 1.34-1.31 (d, J=6.9 Hz, 6H).

### EXAMPLE 16

### 3-[2-fluoro-5-[[4-(2-isopropylthiazole-4-carbonyl)-1-oxa-4,9-diazaspiro[5.5]undecan-9-yl]methyl]phenyl]propanamide

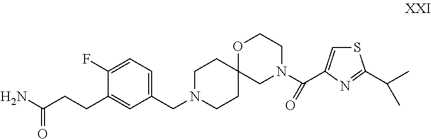 [SMILES: CC(C)c1nc(C(=O)N2CCOC3(CCN(Cc4ccc(F)c(CCC(N)=O)c4)CC3)C2)cs1]

To a solution of 3-[2-fluoro-5-[[4-(2-isopropylthiazole-4-carbonyl)-1-oxa-4,9-diazaspiro[5.5]undecan-9-yl]methyl]phenyl]prop-2-enamide XXII (211.5 g @ 73% w=154.4 g; 317.3 mmoles) in methanol (1.54 L) was added 10% Pd/C (31.72 g, 29.8 mmoles). The mixture was then stirred under hydrogen (4.5 barg) for 12 hours at RT. The reaction mixture was filtered and concentrated in-vacuo to give the title compound XXI (171.0 g @ 87% w=148.8 g; 304.5 mmoles).

¹H NMR (400 MHz, CDCl₃) δ 7.75 (s, 1H), 7.45-7.29 (m, 1H), 7.06-6.98 (m, 1H), 6.91-6.85 (t, J=9.2 Hz, 1H), 5.35-5.22 (m, 1H), 3.95-3.20 (m, 10H), 2.96-2.85 (m, 2H), 2.62-2.31 (m, 6H), 1.86-1.65 (m, 4H), 1.34-1.31 (d, J=6.9 Hz, 6H).

### EXAMPLE 17

### [9-[[3-(2-aminoethyl)-4-fluoro-phenyl]methyl]-1-oxa-4,9-diazaspiro[5.5]undecan-4-yl]-(2-isopropylthiazol-4-yl)methanone dihydrochloride salt

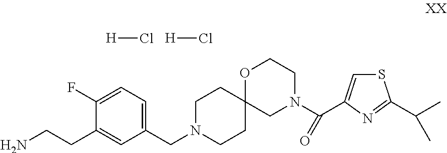

To a solution of 3-[2-fluoro-5-[[4-(2-isopropylthiazole-4-carbonyl)-1-oxa-4,9-diazaspiro[5.5]undecan-9-yl]methyl]phenyl]propanamide XXI (171.0 g @ 87% w=148.8 g; 304.4 mmoles) in acetonitrile (856 mL) was added dropwise a solution of PhI(OOCCF₃)₂ (202.5 g, 457.0 mmoles) in acetonitrile (513 mL) over a period of 20 minutes at 10° C. The resulting mixture was warmed to RT and stirred for 2 hours. A pre-mixed solution of concentrated sulphuric acid (119.4 g) in water (744 mL) was then added to the reaction mixture and stirred for an additional 1 hour. The reaction mixture was then concentrated (to ˜900 mL) and extracted with 2-methyltetrahydrofuran (744 mL then 372 mL). The aqueous layer was collected and basified with aqueous sodium hydroxide solution (10 M; 202 mL). The resulting mixture was extracted twice with 2-methyltetrahydrofuran (402 mL & 342 mL respectively). The aqueous layer was further basified with aqueous sodium hydroxide solution (10 M; 60 mL) before being further extracted with 2-methyltetrahydrofuran (2×342 mL). The combined organic layers were then collected and dried over sodium sulphate. The resulting organic solution was diluted with isopropanol (867 mL) and a solution of HCl in isopropanol (5-6 M; 184 mL) was added. The mixture was then stirred for 16 hours at RT. The resulting solid was collected via filtration and dried in-vacuo at 50° C. to constant weight giving title compound XX as a white solid (97.0 g @ 92% w=89.2 g; 167.0 mmol).

¹H NMR (400 MHz, D₂O) δ 7.67 (s, 0.7H), 7.64 (s, 0.3H), 7.37-7.27 (m, 2H), 7.17-7.11 (m, 1H), 4.81-4.48 (m, 2H), 4.20-4.16 (m, 2H), 3.77-3.42 (m, 7H), 3.29-2.85 (m, 10H), 2.12-2.02 (m, 2H), 1.80-1.75 (bs, 1H), 1.28-1.24 (d, J=6.9 Hz, 6H).

### EXAMPLE 18

### [9-[[3-[2-[benzyl-[(2R)-2-(4-tert-butoxy-2-isopropoxy-1,3-benzothiazol-7-yl)-2-hydroxy-ethyl]amino]ethyl]-4-fluoro-phenyl]methyl]-1-oxa-4,9-diazaspiro[5.5]undecan-4-yl]-(2-isopropylthiazol-4-yl)methanone may be prepared as follows

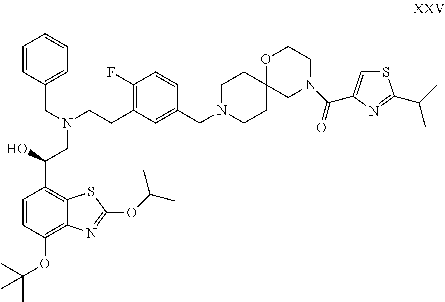 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1]

A mixture of 2-[benzyl-[2-[2-fluoro-5-[[4-(2-isopropylthiazole-4-carbonyl)-1-oxa-4,9-diazaspiro[5.5]undecan-9-yl]methyl]phenyl]ethyl]amino]-1-(4-tert-butoxy-2-isopropoxy-1,3-benzothiazol-7-yl)ethanone XXVI in a suitable alcoholic solvent is hydrogenated using a homochiral transition metal/ligand complex. Filtration and evaporation will yield the title compound XXV in high enantiomeric purity.

### EXAMPLE 19

### 2-[benzyl-[2-[2-fluoro-5-[[4-(2-isopropylthiazole-4-carbonyl)-1-oxa-4,9-diazaspiro[5.5]undecan-9-yl]methyl]phenyl]ethyl]amino]-1-(4-tert-butoxy-2-isopropoxy-1,3-benzothiazol-7-yl)ethanone

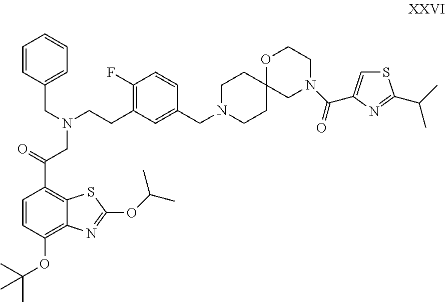 [SMILES: CC(C)Oc1nc2c(OC(C)(C)C)ccc(C(=O)CN(CCc3cc(CN4CCC5(CC4)CN(C(=O)c4csc(C(C)C)n4)CCO5)ccc3F)Cc3ccccc3)c2s1]

A solution of [9-[[3-[2-(benzylamino)ethyl]-4-fluoro-phenyl]methyl]-1-oxa-4,9-diazaspiro[5.5]undecan-4-yl]-(2-isopropylthiazol-4-yl)methanone XXVII (2.18 g, 3.95 mmoles) was dissolved into N-methylpyrrolidinone (12.3 mL) and stirred at RT under nitrogen for 10 minutes. To the resulting solution was added a solution of 1-(4-tert-butoxy-2-isopropoxy-1,3-benzothiazol-7-yl)-2-chloro-ethanone XVI (1.23 g, 3.60 mmoles) in N-methylpyrrolidinone (6.1 mL), followed by diisopropylamine (2.51 mL, 14.4 mmol) and sodium iodide (0.06 g, 0.4 mmol). The mixture was left to stir at RT for 72 hours, resulting in a yellow-orange solution. The mixture was partitioned between water (30 mL) and 2-methyltetrahydrofuran (75 mL). The organic phase was separated and the aqueous phase was extracted 2-methyltetrahydrofuran (2×75 mL). The combined organic phases were then washed with saturated brine solution (75 mL) and evaporated in-vacuo to give a dark-brown oil. Purification by flash column chromatography (0-2% methanol in ethyl acetate) and evaporation gave title compound XXVI as a white solid (1.90 g; 2.21 mmol).

¹H NMR (500 MHz, d₆-DMSO) δ 8.06-7.87 (m, 2H), 7.37-6.87 (m, 9H), 5.33 (dt, J=6.2, 12.2 Hz, 1H), 4.12 (s, 7H), 3.86-3.41 (m, 4H), 3.40-3.19 (m, 3H), 2.75 (d, J=48.1 Hz, 3H), 2.43-2.01 (m, 4H), 1.75-1.06 (m, 25H).

### EXAMPLE 20

### [9-[[3-[2-tert-butoxyvinyl]-4-fluoro-phenyl]methyl]-1-oxa-4,9-diazaspiro[5.5]undecan-4-yl]-(2-isopropylthiazol-4-yl)methanone

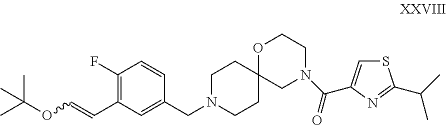 [SMILES: CC(C)c1nc(C(=O)N2CCOC3(CCN(Cc4ccc(F)c(C=COC(C)(C)C)c4)CC3)C2)cs1]

### Method 1

[9-[(3-bromo-4-fluoro-phenyl)methyl]-1-oxa-4,9-diazaspiro[5.5]undecan-4-yl]-(2-isopropylthiazol-4-yl)methanone XXIII (50 mg; 94 μmoles) was slurried in N-methylpyrrolidinone (1.0 mL). To the slurry was added dicyclohexylmethyl amine (60 μL; 282 μmoles), tert-butylvinyl ether (49 μL; 375 μmoles) and Pd-116 (6.2 mg; 9.4 μmoles). The mixture was stirred at RT for 3 days. After this time the reaction was diluted with water and extracted with organic solvent to yield a solution of the product along with its Z-isomer and the α-regioisomer.

### Method 2

[9-[(3-bromo-4-fluoro-phenyl)methyl]-1-oxa-4,9-diazaspiro[5.5]undecan-4-yl]-(2-isopropylthiazol-4-yl)methanone XXIII (50 mg; 94 μmoles) was added to tetrabutylammonium bromide (500 mg; 1550 μmoles). To the solid mixture was added tetrabutylammonium acetate (85 mg; 282 μmoles), tert-butylvinyl ether (49 μL; 375 μmoles) and palladium acetate (1.1 mg; 4.7 μmoles). The reaction was heated in a sealed vessel at 90° C. with vigorous stirring. At this temperature the reaction was a mobile solution. After 18 hours the reaction was diluted with water and extracted with organic solvent. The organic phase was back extracted several times with water, yielding a solution of the product along with its Z-isomer and the α-regioisomer.

### EXAMPLE 21

### 2-[2-fluoro-5-[[4-(2-isopropylthiazole-4-carbonyl)-1-oxa-4,9-diazaspiro[5.5]undecan-9-yl]methyl]phenyl]acetaldehyde may be prepared as follows

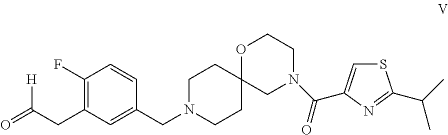 [SMILES: CC(C)c1nc(C(=O)N2CCOC3(CCN(Cc4ccc(F)c(CC=O)c4)CC3)C2)cs1]

A solution of [9-[[3-[2-tert-butoxyvinyl]-4-fluoro-phenyl]methyl]-1-oxa-4,9-diazaspiro[5.5]undecan-4-yl]-(2-isopropylthiazol-4-yl)methanone is treated in an analogous manner to its methyl analogue, [3-[[4-fluoro-3-[2-methoxyvinyl]phenyl]methyl]-7-oxa-3,10-diazaspiro[5.5]undecan-10-yl]-(2-isopropylthiazol-4-yl)methanone VII, to obtain a solution of the title compound for use in downstream chemistry.

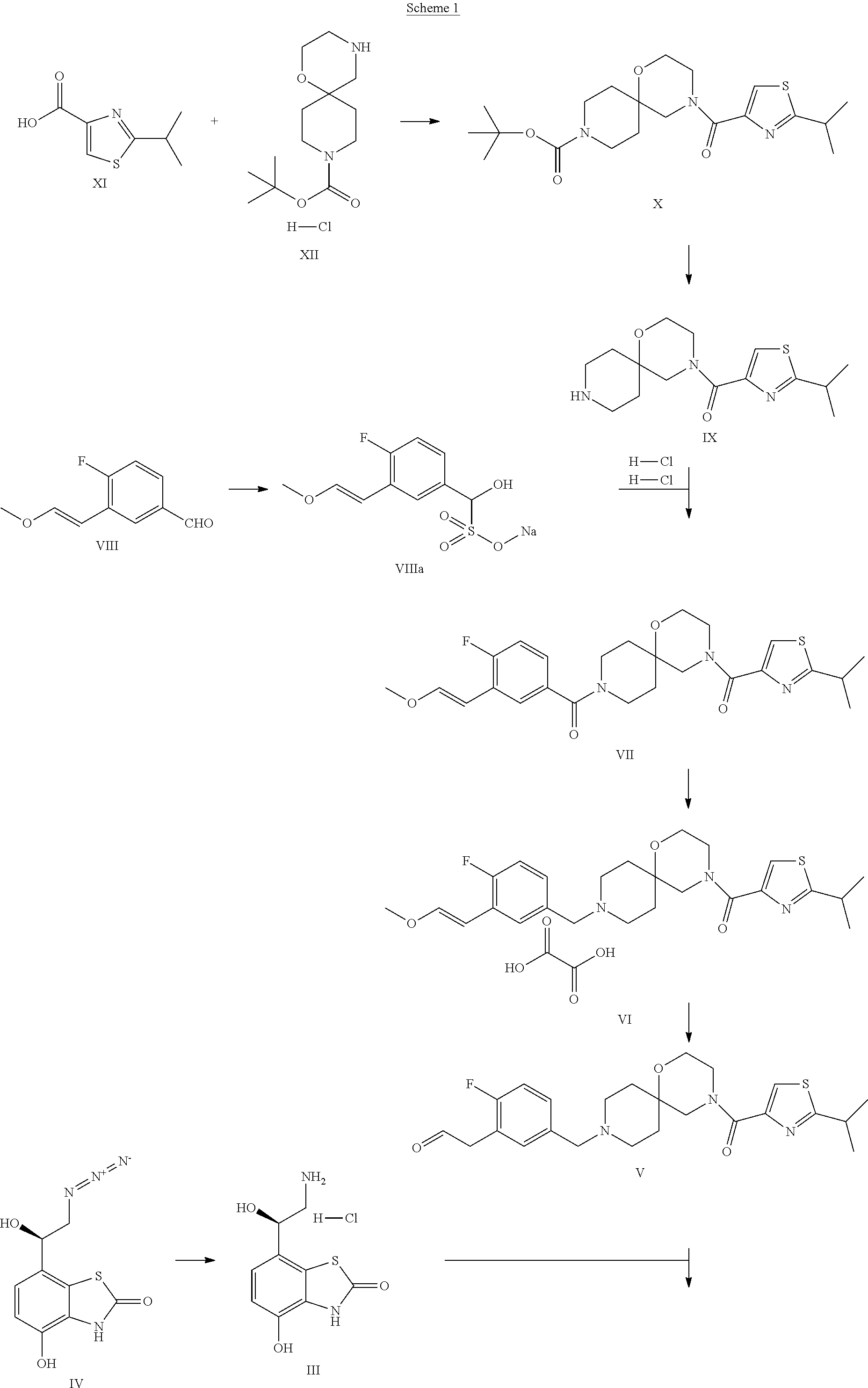

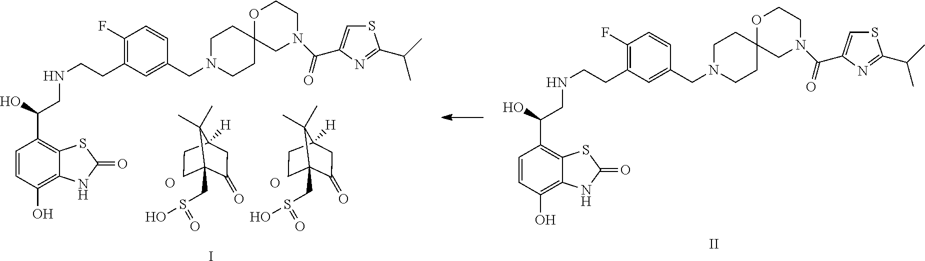

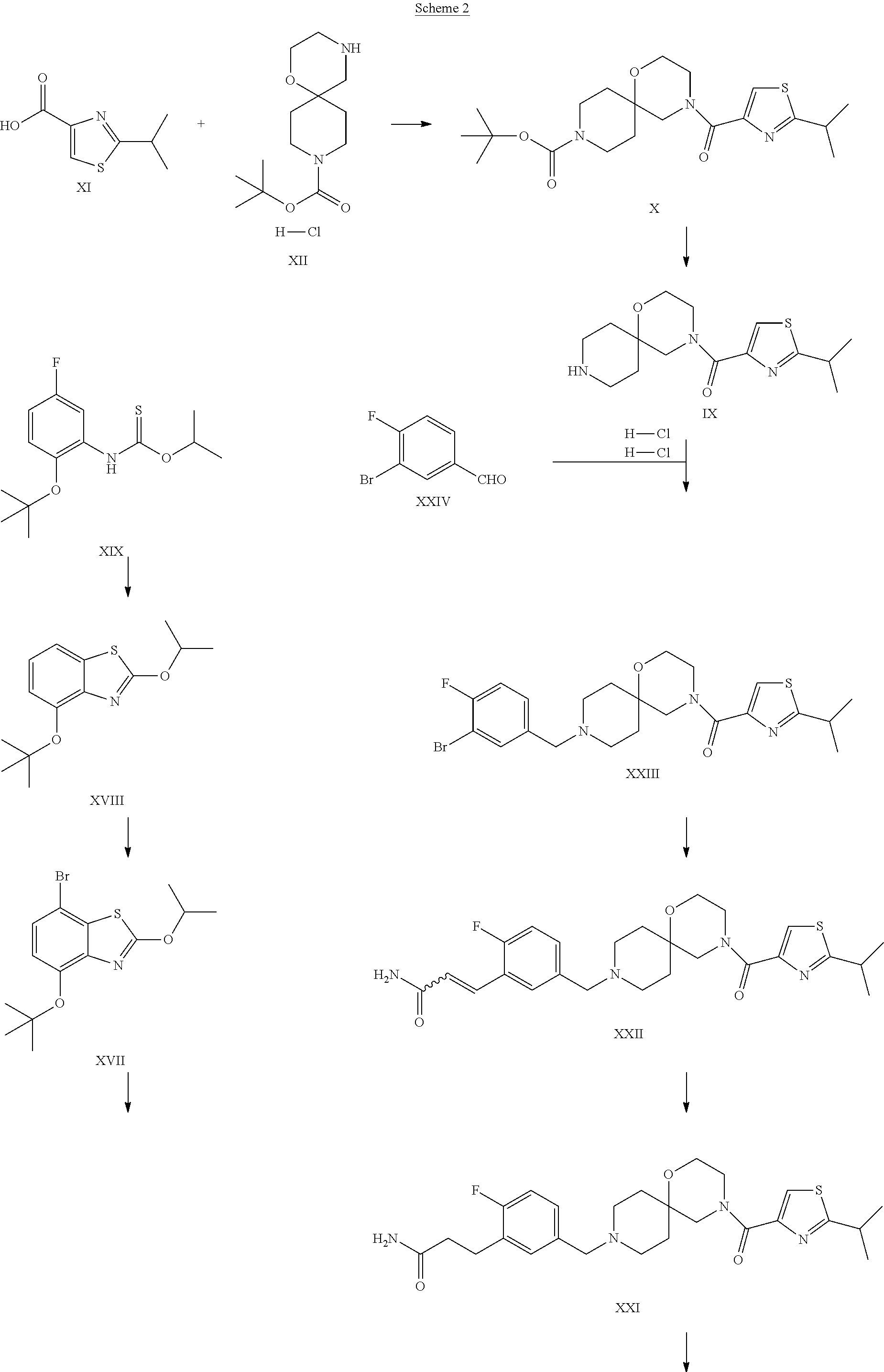

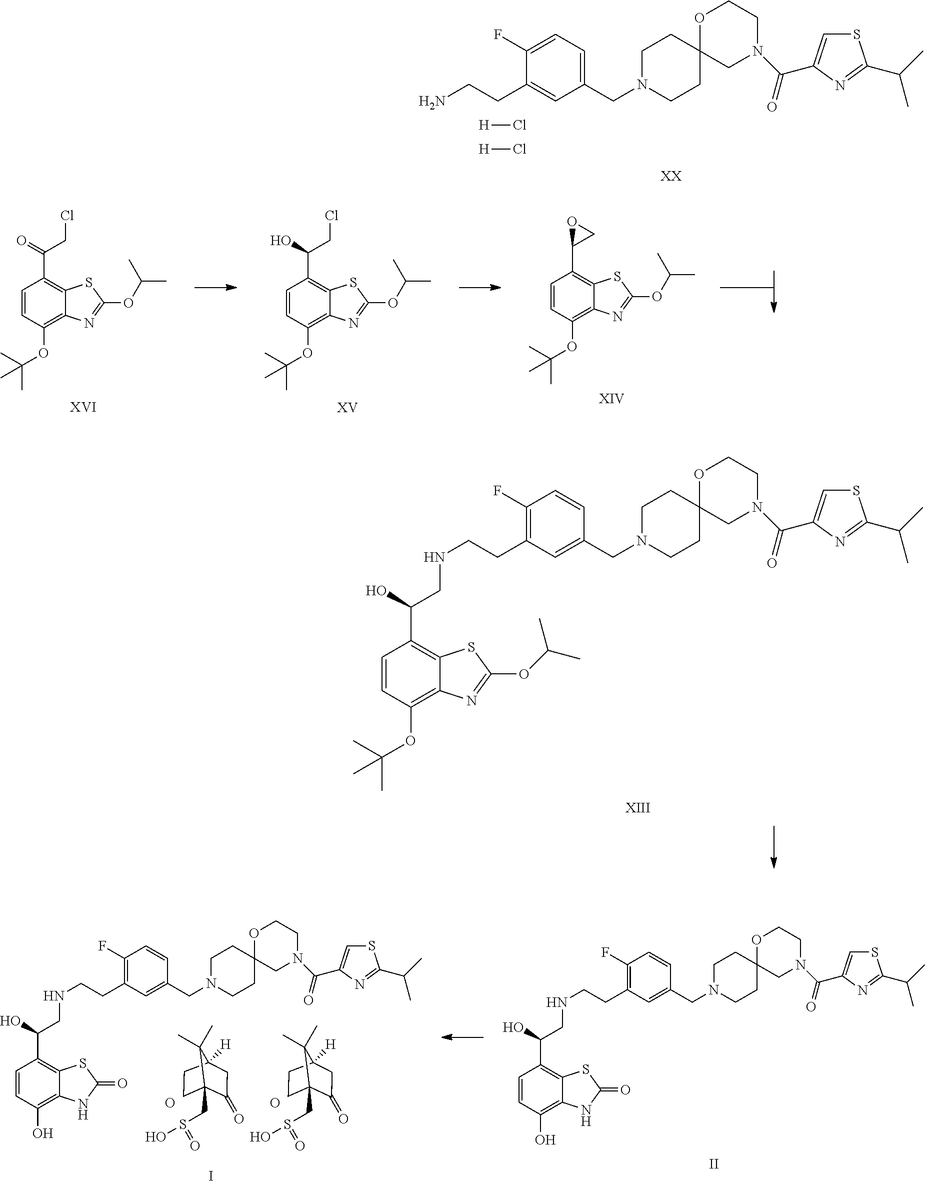

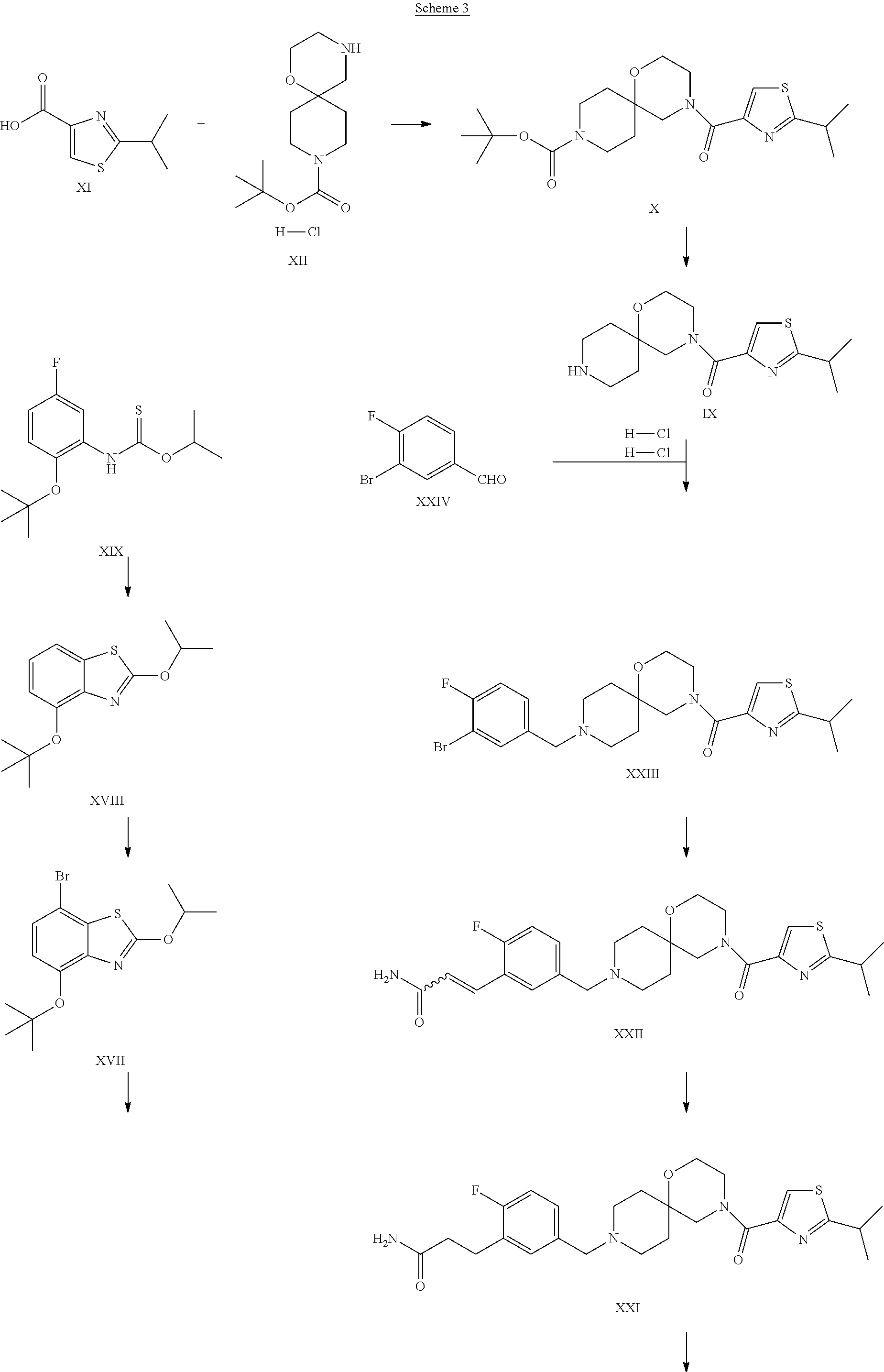

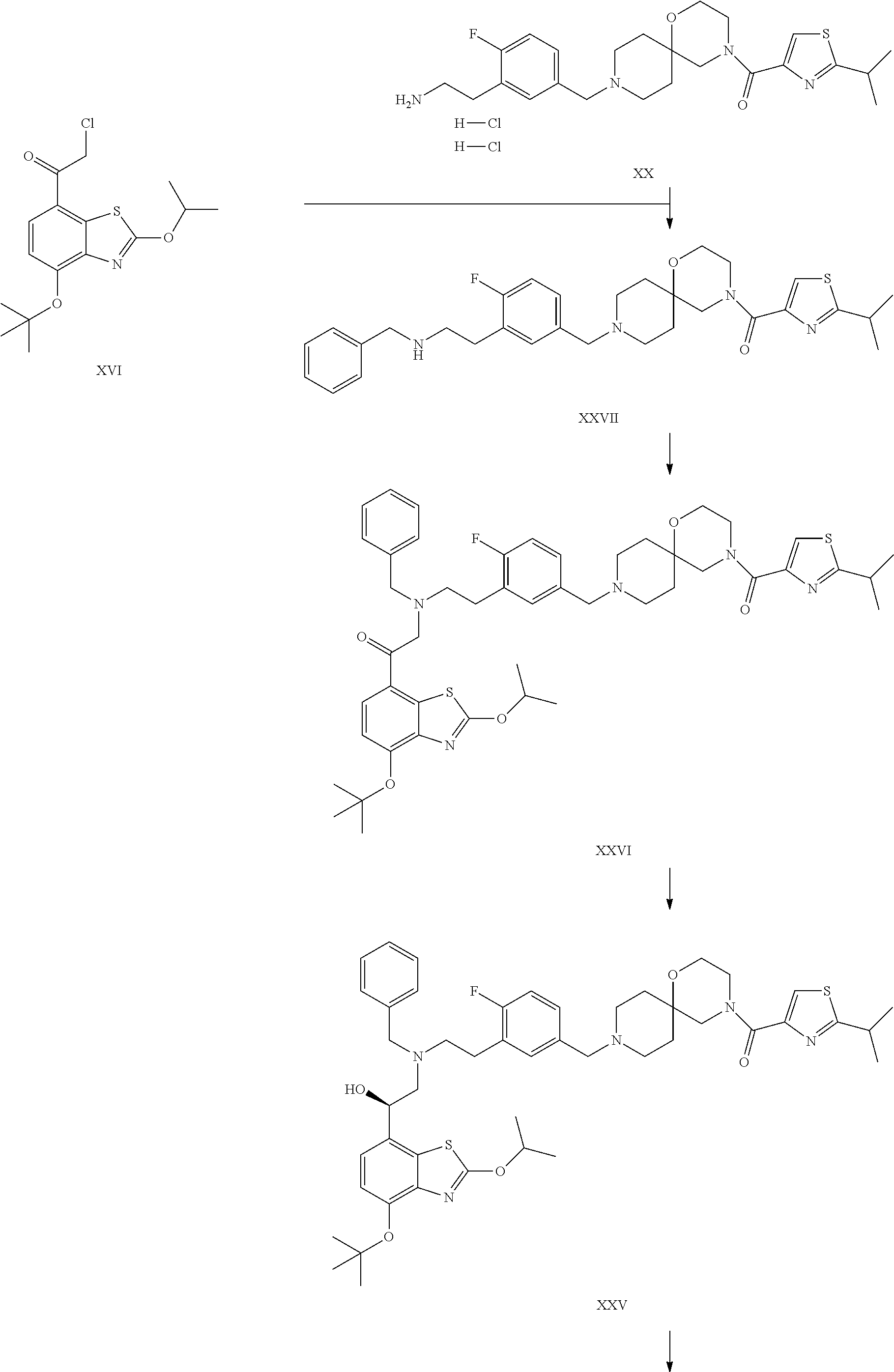

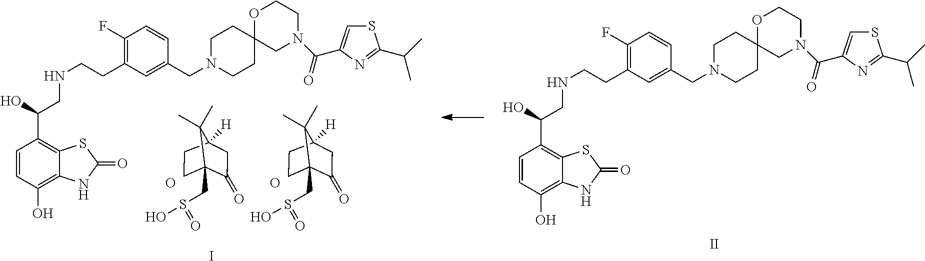

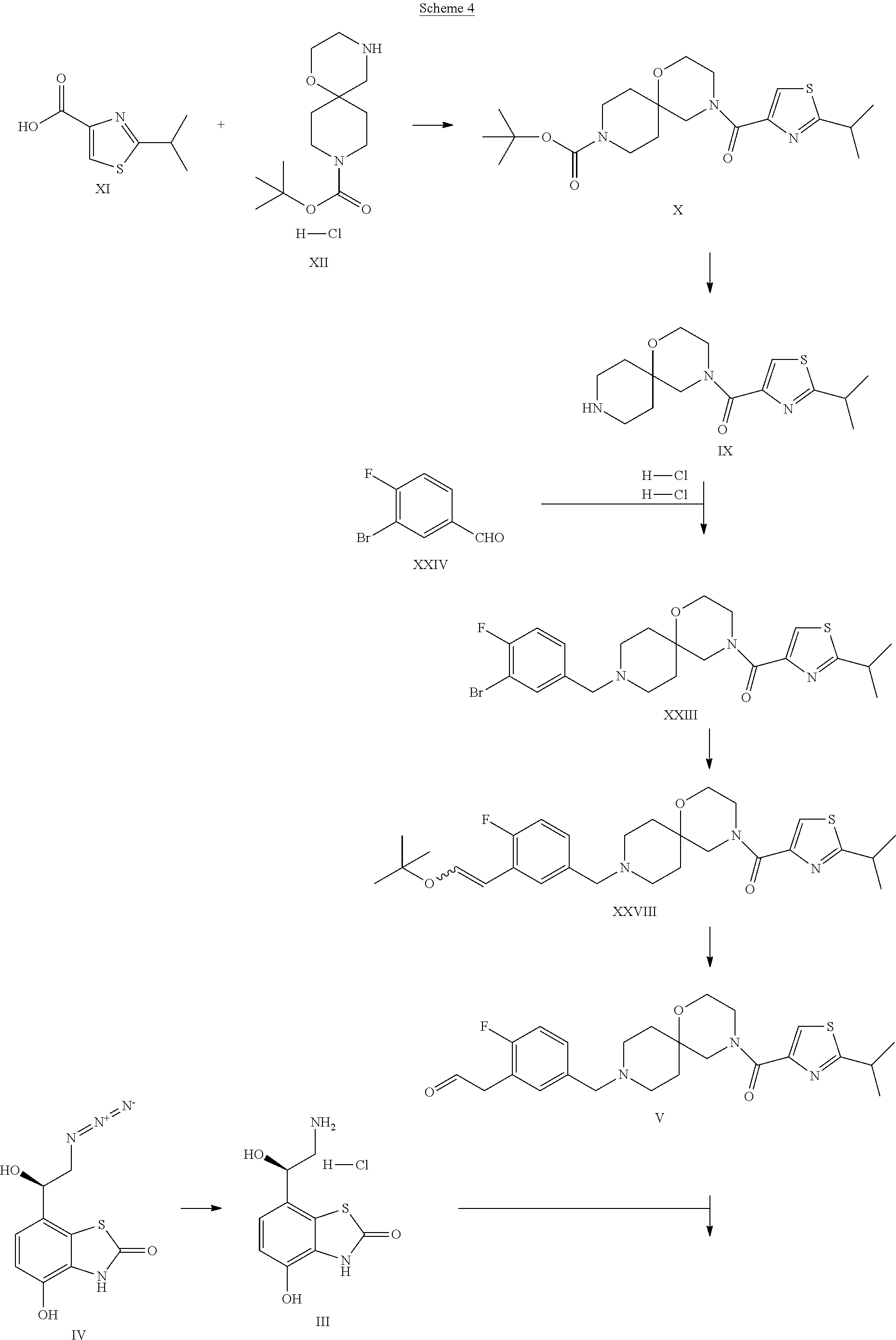

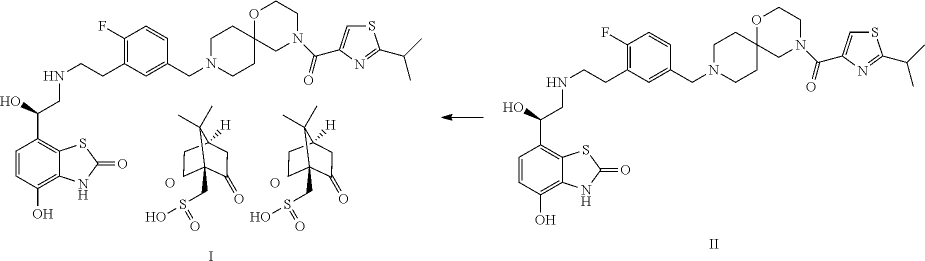

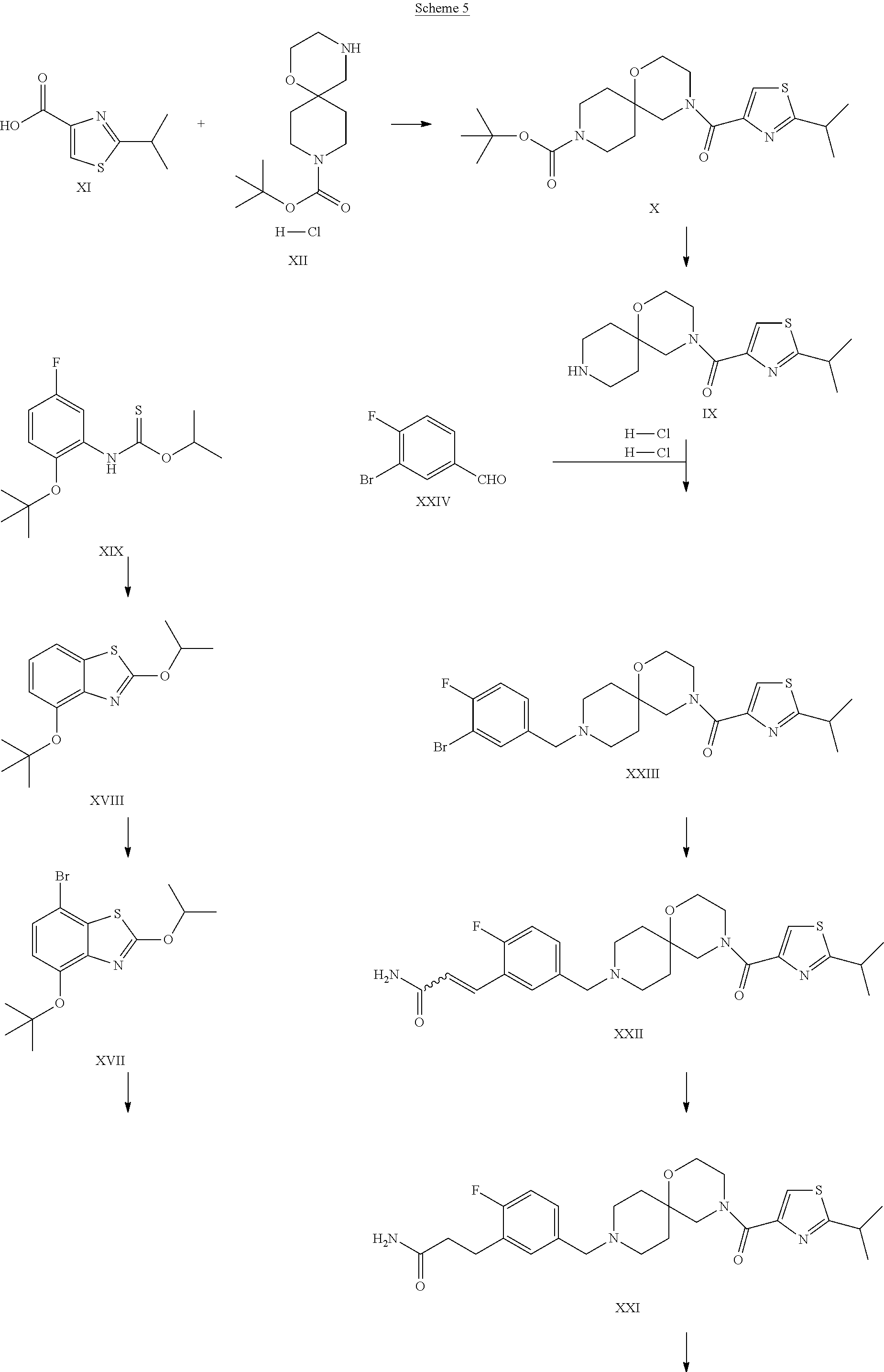

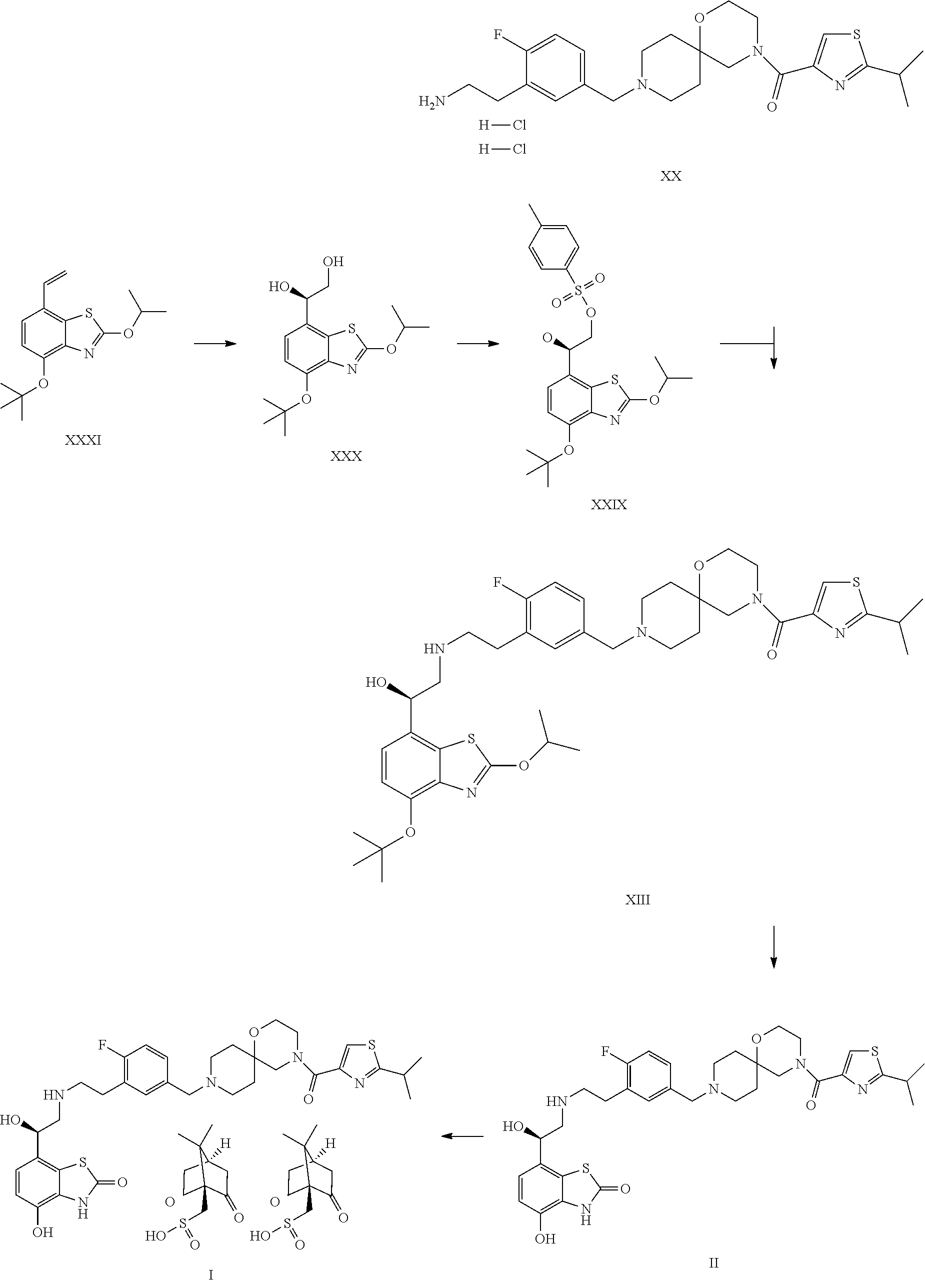

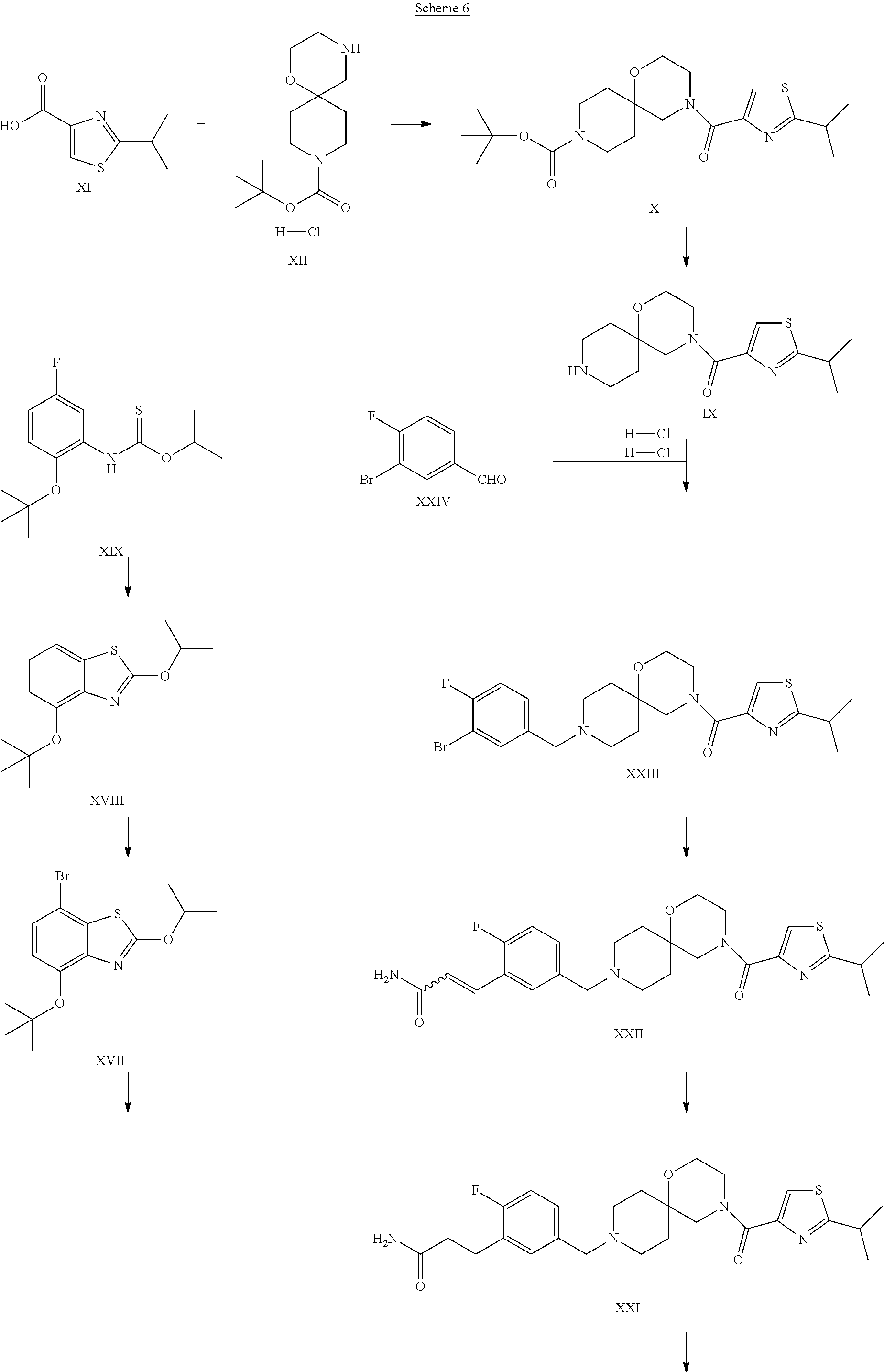

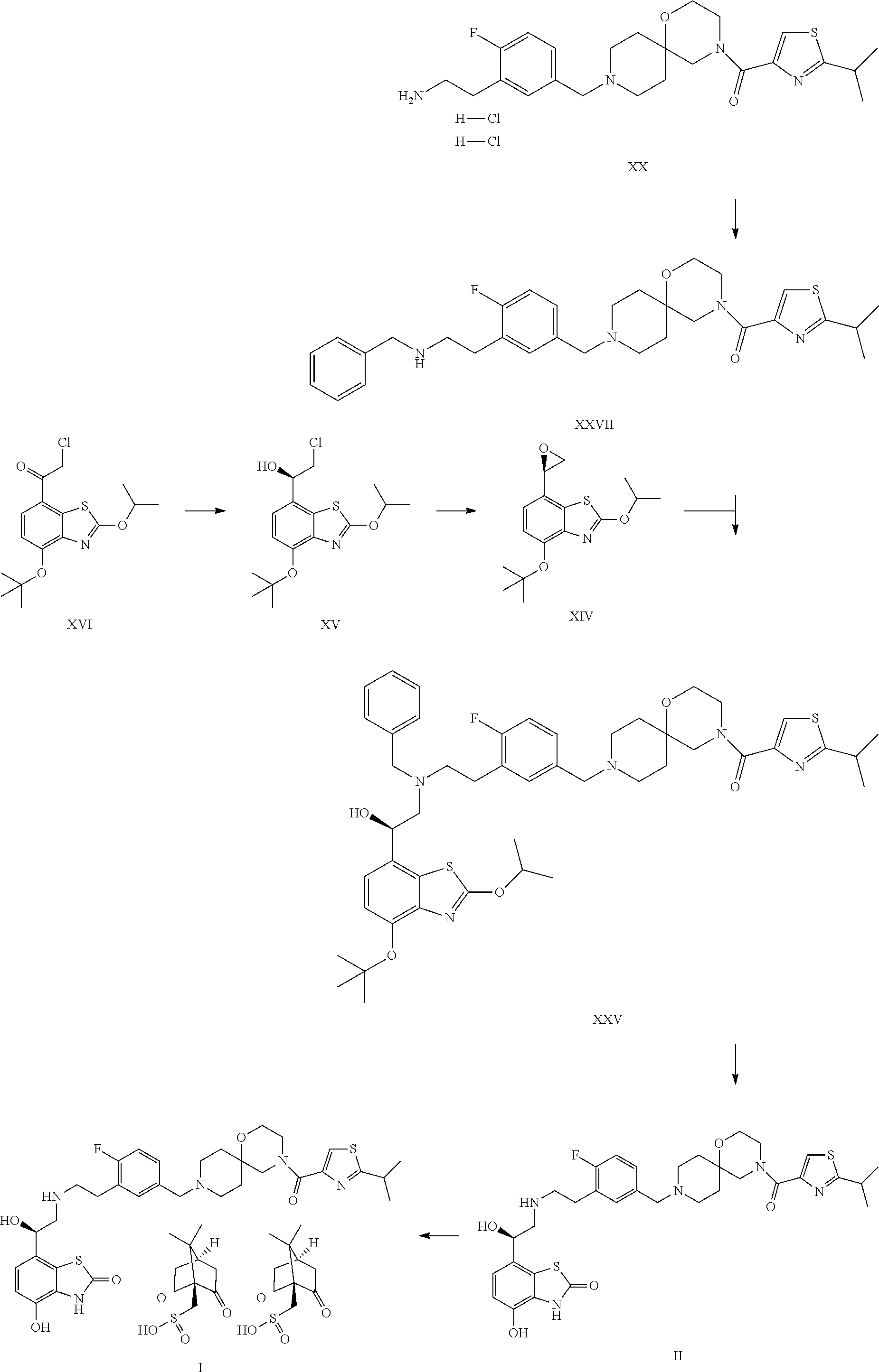

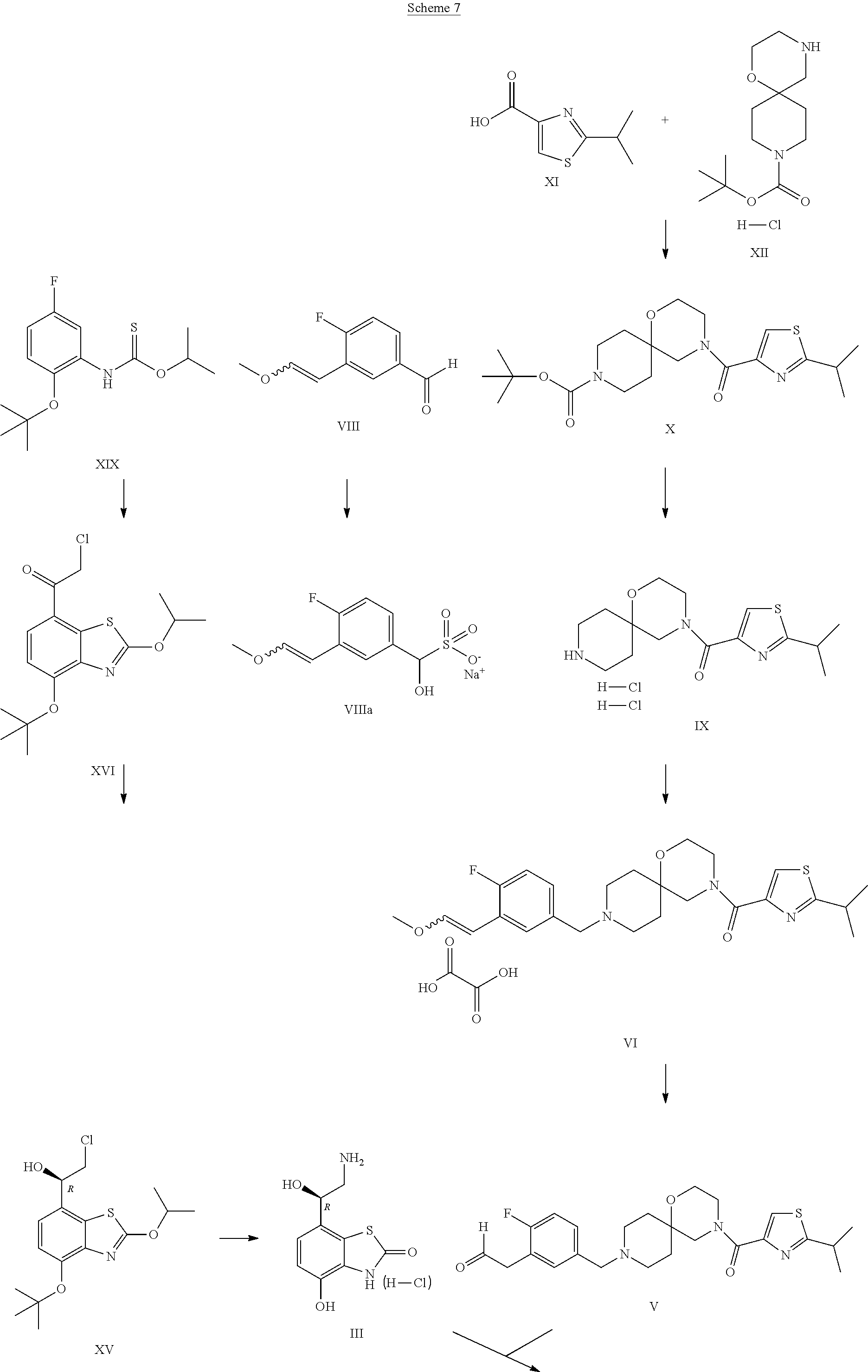

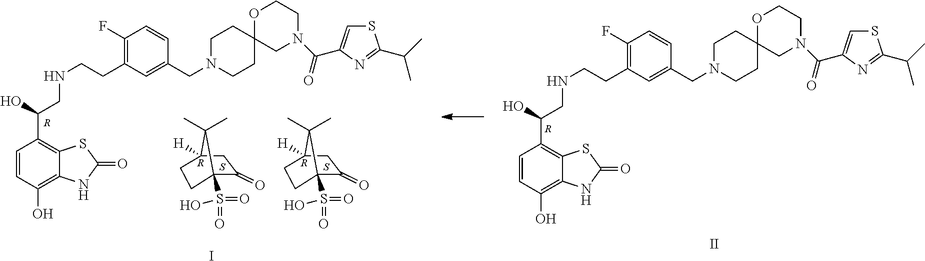

1. A process for the preparation of the compound of formula II 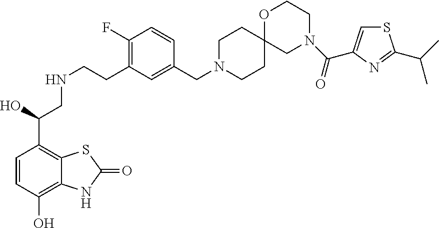 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]
and pharmaceutically acceptable salts thereof which process comprises reaction of the compound of formula III or alternate salt thereof 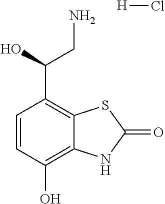 [SMILES: Cl ; NC[C@H](O)c1ccc(O)c2[nH]c(=O)sc12]
and the compound of formula V 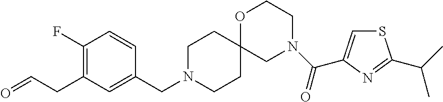 [SMILES: CC(C)c1nc(C(=O)N2CCOC3(CCN(Cc4ccc(F)c(CC=O)c4)CC3)C2)cs1]
in a suitable solvent and at a suitable temperature under reductive conditions comprising hydrogen in the presence of a metal catalyst so as to give the compound of formula II followed by conversion to a pharmaceutically acceptable salt as required.

2. A process for the preparation of the compound of formula II 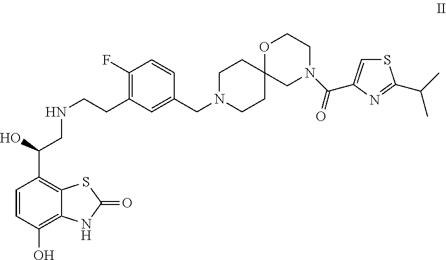 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]
which process comprises reaction of the compound of formula XX 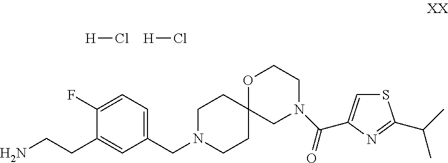
or any other suitable alternate salt (or the neutral, parent amine) there of with the compound of formula XIV 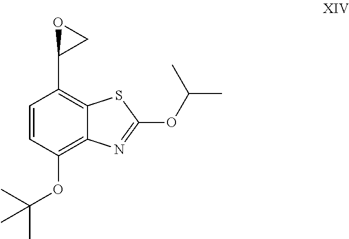 [SMILES: CC(C)Oc1nc2c(OC(C)(C)C)ccc([C@@H]3CO3)c2s1]
in a suitable solvent and in the presence of a base (not required when using the neutral, parent amine XX) to give the compound of formula XIII 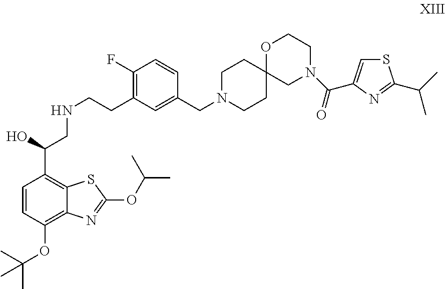 [SMILES: CC(C)Oc1nc2c(OC(C)(C)C)ccc([C@@H](O)CNCCc3cc(CN4CCC5(CC4)CN(C(=O)c4csc(C(C)C)n4)CCO5)ccc3F)c2s1]
followed by deprotection so as to give the compound of formula II.

3. A process for the preparation of the compound of formula II 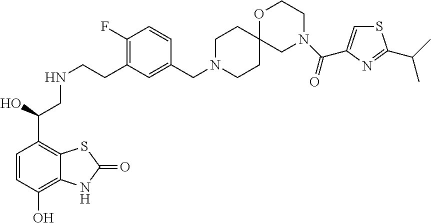 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]
and pharmaceutically acceptable salts thereof which process comprises reacting the compound of formula XVI 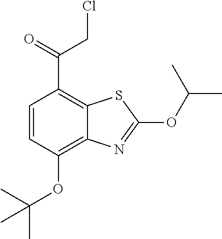 [SMILES: CC(C)Oc1nc2c(OC(C)(C)C)ccc(C(=O)CCl)c2s1]
with the compound of formula XXVII 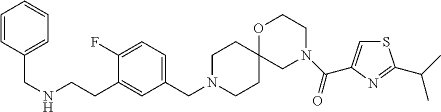 [SMILES: CC(C)c1nc(C(=O)N2CCOC3(CCN(Cc4ccc(F)c(CCNCc5ccccc5)c4)CC3)C2)cs1]
in a suitable solvent in the presence of a base and a source of iodide to give the compound of formula XXVI 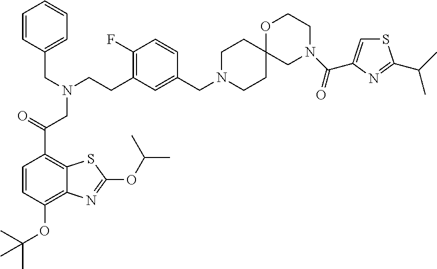 [SMILES: CC(C)Oc1nc2c(OC(C)(C)C)ccc(C(=O)CN(CCc3cc(CN4CCC5(CC4)CN(C(=O)c4csc(C(C)C)n4)CCO5)ccc3F)Cc3ccccc3)c2s1]
which is then reduced in a suitable alcoholic solvent under transfer hydrogenation conditions and using a homochiral transition metal/ligand complex to give the compound of formula XXV 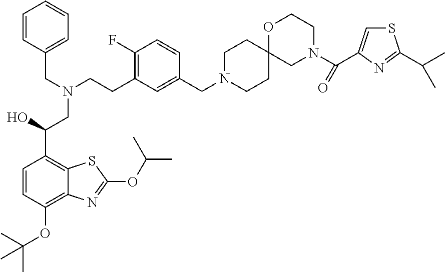 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1]
which is then deprotected in a suitable solvent in the presence of a metal catalyst for example palladium black so as to give the compound of formula II followed by conversion to a pharmaceutically acceptable salt as required.

4. A process for the preparation of the compound of formula II 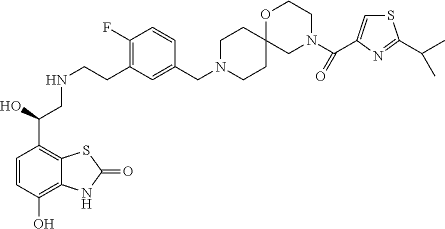 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]
and pharmaceutically acceptable salts thereof which process comprises reaction of a compound of formula XXIII 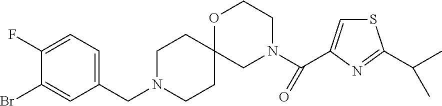 [SMILES: CC(C)c1nc(C(=O)N2CCOC3(CCN(Cc4ccc(F)c(Br)c4)CC3)C2)cs1]
in a suitable solvent, by the addition of t-butylvinyl ether; a metal catalyst or ligand/phase transfer catalyst/base combination to give a compound of formula XXVIII 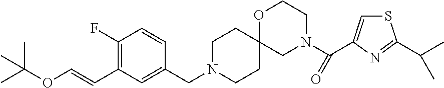 [SMILES: CC(C)c1nc(C(=O)N2CCOC3(CCN(Cc4ccc(F)c(/C=C/OC(C)(C)C)c4)CC3)C2)cs1]
which is then converted to a compound of formula V 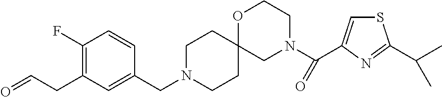 [SMILES: CC(C)c1nc(C(=O)N2CCOC3(CCN(Cc4ccc(F)c(CC=O)c4)CC3)C2)cs1]
via addition to a suitable acid which is then reacted with the compound of formula III 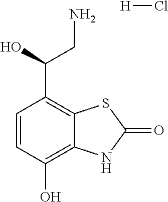 [SMILES: Cl ; NC[C@H](O)c1ccc(O)c2[nH]c(=O)sc12]
or any alternative salt thereof, in a suitable solvent under hydrogenation conditions in the presence of a metal catalyst or borane based reducing agent
so as to give the compound of formula II followed by conversion to a pharmaceutically acceptable salt as required

5. A process for the preparation of the compound of formula II 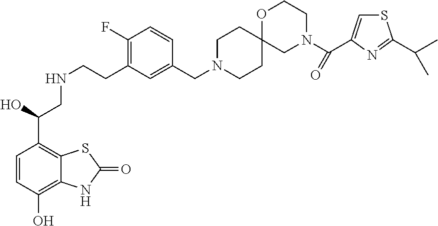 [SMILES: CC(C)c1nc(C(=O)N2CCOC3(CCN(Cc4ccc(F)c(CCNC[C@H](O)c5ccc(O)c6[nH]c(=O)sc56)c4)CC3)C2)cs1]
and pharmaceutically acceptable salts thereof which process comprises reaction of a compound of formula XX or alternate salt thereof 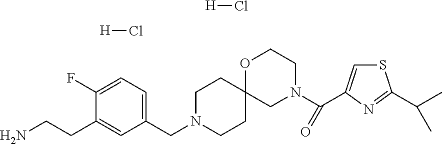 [SMILES: Cl ; Cl ; CC(C)c1nc(C(=O)N2CCOC3(CCN(Cc4ccc(F)c(CCN)c4)CC3)C2)cs1]
with the compound of formula XXIX 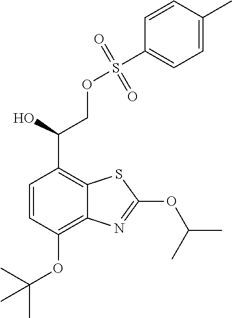 [SMILES: Cc1ccc(S(=O)(=O)OC[C@H](O)c2ccc(OC(C)(C)C)c3nc(OC(C)C)sc23)cc1]
in a suitable solvent and in the presence of a base to give a compound of the compound of formula XIII 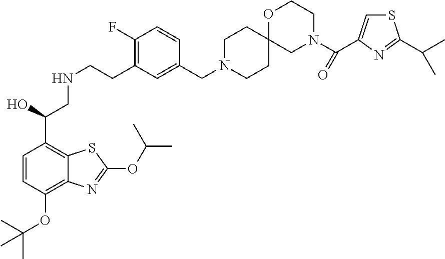 [SMILES: CC(C)Oc1nc2c(OC(C)(C)C)ccc([C@@H](O)CNCCc3cc(CN4CCC5(CC4)CN(C(=O)c4csc(C(C)C)n4)CCO5)ccc3F)c2s1]
followed by deprotection to give a compound of formula II followed by conversion to a pharmaceutically acceptable salt as required

6. A process for the preparation of the compound of formula II and pharmaceutically acceptable salts thereof which process comprises reacting the compound of formula XXVII 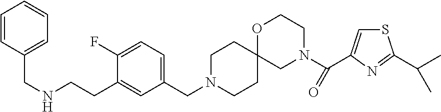 [SMILES: CC(C)c1nc(C(=O)N2CCOC3(CCN(Cc4ccc(F)c(CCNCc5ccccc5)c4)CC3)C2)cs1]
with the compound of formula XIV 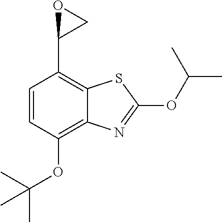 [SMILES: CC(C)Oc1nc2c(OC(C)(C)C)ccc([C@@H]3CO3)c2s1]
in a suitable solvent and base to give the compound of formula XXV 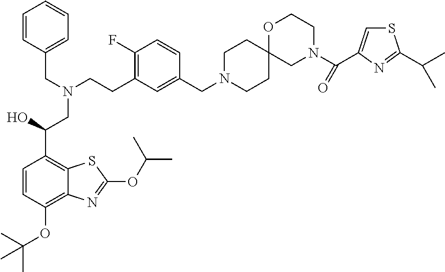 [SMILES: CC(C)Oc1nc2c(OC(C)(C)C)ccc([C@@H](O)CN(CCc3cc(CN4CCC5(CC4)CN(C(=O)c4csc(C(C)C)n4)CCO5)ccc3F)Cc3ccccc3)c2s1]
followed by deprotection, to give a compound of formula II and followed by conversion to a pharmaceutically acceptable salt as required.

7. A process for the preparation of the compound of formula II and pharmaceutically acceptable salts thereof which comprises reaction of the compound of formula III or any other suitable alternate salt there of 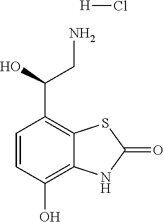
and the compound of formula V

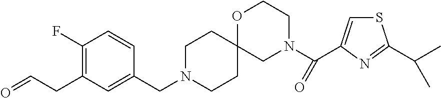 [SMILES: CC(C)c1nc(C(=O)N2CCOC3(CCN(Cc4ccc(F)c(CC=O)c4)CC3)C2)cs1]

8. A novel intermediate compound as set out in Table 1 hereinbefore.
